(19) United States
(12) Patent Application Publication
(10) Pub. No.: US 2005/0095591 A1
(43) Pub. Date: May 5, 2005

(54) Diagnostic assay

(76) Inventors: Richard Ian Christopherson, Paddington (AU); Christobal Guillermo Dos Remedios, Paddington (AU); David Stephen Celermajer, Vaucluse (AU)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 10/489,623

(22) PCT Filed: Sep. 12, 2001
(86) PCT No.: PCT/AU01/01141

(30) Foreign Application Priority Data
Sep. 12, 2000 (AU) PR 0056

(51) Int. Cl. C12Q 1/68

(52) U.S. Cl. 435/6

(57) ABSTRACT

The present invention relates generally to a diagnostic device including a prognostic assay for parameters which are indicative of a condition or event associated with the systemic vasculature. More particularly, the present invention provides an assay to detect parameters associated with a vascular disease including cardiovascular, stroke, pulmonary, renovascular, cerebrovascular, thrombotic or generalized arterial or venous condition or event including acute coronary syndrome such as but not limited to acute myocardial infarction, heart failure, atheromoma or a thrombotic condition. The identification of these parameters or more particularly a pattern of parameters enables the diagnosis of a condition or event or the determination of the risk of development of a condition or event associated to the systemic vasculature. Still more particularly, the present invention is directed to a diagnostic device comprising a set of members wherein one or more of said members has or have specific or generic binding partners in a biological sample from an animal including human subject wherein the pattern of binding of the members to the binding partners is indicative, predictive or otherwise associated with a likelihood of a condition or event within the systemic vasculature. The absence of detection of specific or generic binding partners is also of indicative or predictive value. This is particularly important in cases where patients are unable to communicate advice to a physician on their own condition, such as during surgery or for patients in a coma. It is also useful in determining the risk of a vascular disease including cardiovascular, stroke, pulmonary, renovascular, cerebrovascular, thrombotic or generalized arterial or venous conditions or events in a healthy subject or a subject entering into an exposure to risk such as surgery or chemotherapy. The present invention is useful inter alia for the identification and/or quantitation of biochemical markers of conditions or events in the systemic vasculature such as heart disease, heart disorders, infections of the heart, stroke and thrombosis as well as the determination of a risk of development of these conditions including the absence of disorders or absence of risk of the development of a disorder. The assessment of such conditions may be made in a clinical setting, as part of triage, as part of a routine testing protocol and/or as a laboratory procedure.

### FIELD OF THE INVENTION

The present invention relates generally to a diagnostic device including a prognostic assay for parameters which are indicative of a condition or event associated with the systemic vasculature. More particularly, the present invention provides an assay to detect parameters associated with a vascular disease including cardiovascular, stroke, pulmonary, renovascular, cerebrovascular, thrombotic or generalized arterial or venous condition or event including acute coronary syndrome such as but not limited to acute myocardial infarction, heart failure, atheromoma or a thrombotic condition. The identification of these parameters or more particularly a pattern of parameters enables the diagnosis of a condition or event or the determination of the risk of development of a condition or event associated to the systemic vasculature. Still more particularly, the present invention is directed to a diagnostic device comprising a set of members wherein one or more of said members has or have specific or generic binding partners in a biological sample from an animal including a human subject wherein the pattern of binding of the members to the binding partners is indicative, predictive or otherwise associated with a likelihood of a condition or event within the systemic vasculature. The absence of detection of specific or generic binding partners is also of indicative or predictive value. This is particularly important in cases where patients are unable to communicate advice to a physician on their own condition, such as during surgery or for patients in a coma. It is also useful in determining the risk of vascular disease including cardiovascular, stroke, pulmonary, renovascular, cerebrovascular, thrombotic or generalized arterial or venous conditions or events in a healthy subject or a subject entering into an exposure to risk such as surgery or chemotherapy. The present invention is useful inter alia for the identification and/or quantitation of biochemical markers of conditions or events in the systemic vasculature such as heart disease, heart disorders, infections of the heart, stroke and thrombosis as well as the determination of a risk of development of these conditions including the absence of disorders or absence of risk of the development of a disorder. The assessment of such conditions may be made in a clinical setting, as part of triage, as part of a routine testing protocol and/or as a laboratory procedure.

### BACKGROUND OF THE INVENTION

Bibliographic details of the publications referred to by author in this specification are collected at the end of the description.

Reference to any prior art in this specification is not, and should not be taken as, an acknowledgment or any form of suggestion that this prior art forms part of the common general knowledge in Australia or any other country.

Cardiovascular disease such as coronary arterial disease and acute myocardial infarctions (AMI) are examples of conditions or events associated with the systemic vasculature. Cardiovascular disease itself affects over 70 million people in the Western World. AMI is the largest contributor to cardiovascular disease affecting approximately 1.5 million patients per year. Patients with AMI fall into one of two categories.

A first category comprises approximately 20-30% of patients and is characterized by “silent” infarctions. Such infarctions are “silent” as patients are frequently unaware of the infarction due to an absence of pain. A second category comprises the remaining two thirds of patients and represents those patients who present to physicians with severe chest pain.

The diagnosis of either category of AMI requires a series of induced tests requiring multiple independent blood-based analyses to determine potential myocardial tissue damage. Typical AMI is associated with severe and prolonged chest pain, otherwise referred to as angina. Current diagnosis involves changes in electrocardiograms (ECG) usually including elevated S-T segments where there are Q or QS waves and changes in serum enzyme levels.

Silent ischemia and infarction are also important clinical problems. Breathlessness can be due to an infarction and there is a need to rapidly assess cardiac damage when this occurs. However, detection of silent infarctions can be problematic. It may be induced by exercise where ECG performance is monitored by continuous ECG monitoring over 24 hours. Depression (>1 mm) of the ST segment of an ECG is diagnostic but about one third of AMI patients have apparently normal ECGs. Alternatively, angiographic screening can be performed. The detection of small infarcts (which commonly contribute to the pool of silent infarcts) is limited by the comparatively poor sensitivity of some biochemical markers. There is an increased incidence of mortality associated with small infarcts. Determination of the MB isoform of creatine kinase (CK-MB) is a particularly useful marker of silent infarcts but is generally not correlated with a range of other markers.

The causes of AMI can be multiple and they are not clearly understood. A recent impetus was given to the concept that AMI is an inflammatory response by the heart to an infection that may be evident elsewhere such as a periodontal infection (Herzberg & Meyer, 1998), but which can manifest itself in the heart.

Atherosclerosis and vascular endothelial injury can be the result of an immunological response by the patient to proteins produced by microbiological infections. Heat shock proteins (HSPs) from microorganisms such as Escherichia coli and Chlamydia are highly conserved and result in immunological cross-reactions with related human proteins expressed by cells in the cardiovascular system such as endothelial cells (Mayr et al, 1999).

As stated above, cardiovascular conditions are one example of conditions and events involving the systemic vasculature. The systemic vasculature comprises inter alia capillaries, veins, arteries, arterioles, venuoles, the heart and other organs. A range of conditions such as renal failure, thrombosis, organ transplant rejection, infection and vascularization of tumorous and cancerous growths are associated with the systemic vasculature.

There is a need to develop an assay which simultaneously determines multiple parameters required to assess a condition or event of the systemic vasculature. The term “multiple” in this context means two or more. The present inventors have developed an assay useful in determining the risk of a condition or event arising, the presence of a previous event or condition as well as post-operative prognoses and future events in a subject including a human.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The present invention is predicated in part on the use of multiple markers or parameters to assist in the diagnosis or prognosis of a particular condition or event associated with the systemic vasculature. Examples where such diagnoses or prognoses are useful is in assessing healthy and unhealthy animals including human subjects, diagnosing particular conditions or events such as a vascular disease including cardiovascular, stroke, pulmonary, renovascular, cerebrovascular, thrombotic or generalized arterial or venous condition or event or renal or heart failure and in determining the risk of development of such a condition or event in, for example, healthy subjects or subjects to be exposed to certain risks such as inter alia surgery, a course of therapy or vaccination.

Accordingly, one aspect of the present invention contemplates a method of assessing the parameters associated with a condition or event of the systemic vasculature or assessing a risk of a condition or event occurring, said method comprising obtaining a biological sample from a subject to be tested wherein said biological sample comprises one or more members which are present, absent, elevated or otherwise activated or up or down regulated in a subject prior to, during or following said condition or event and contacting said biological sample with a second set of members wherein one or more of said second set of members are binding partners to one or more of said first set of members and wherein the pattern of interaction between said first and second sets of members including the absence of interaction is indicative of said condition or event or the risk of development of same.

Another aspect of the present invention contemplates a method of assessing the parameters associated with a condition or event associated with the systemic vasculature, said method comprising contacting a biological sample from a subject to be tested wherein said biological sample comprises one or more members which are present, absent, elevated or otherwise activated in a subject following said condition or event, said members being selected from two or more of myoglobin, myosin light chain (IC), myosin heavy chain (MHC), total creatine kinase (CK) including CK-MB, lactate dehydrogenase (LDH-H4), aspartate aminotransferase (AST), cardiac troponin I and T (cTn-I and cTn-T, respectively) and cTn-I and cTn-I RNA, fatty acid binding protein (FAB protein) including FABP1 and human heart-type, glycogen phosphorylase-BB isoenzyme, α-atrial natriuretic peptide (ANP), cytoplasmic FABP, brain natriuretic peptide (BNP), adrenomedullin (ADM), low density lipoprotein (LDL), very low density lipoprotein (VLDL), high density lipoprotein (HDL) and intermediate density lipoprotein (IDL), C reactive protein (CRP), serum amyloid A, P-selectin, prostaglandins, platelet-activating factor (PAF), histamine, tumor necrosis factor α (TNFα), soluble TNF receptor 2 (sTNFR2), fibrin, fibrinogen, fibrinolytic peptides, modified haemoglobin (HbA1c), ferritin, soluble intercellular adhesion molecule (ICAM) including soluble intercellular adhesion molecule-1 (ICAM1), heat shock proteins, apoB, apoA, apoE, homocysteine or parts thereof, Streptococcus sp., Porphyromonas gingivalis, Helicobacter pylori and Chlamydia pneumoniae or immunological relatives thereof, necrosis and platelet markers, leptin, vasopeptidase inhibitor of cardiac endogenous kinins, heparin, metalloproteinase-9, metalloproteinase-1 including its tissue inhibitor, angiotensin-converting enzyme, CD95/Apo1/Fas, hepatocyte growth factor, soluble vascular cell adhesion molecule-1 (VCAM1), plasma brain natriuretic peptide, angiotensin II type receptor, endothelial constitutive nitric oxide synthase, glycoprotein IIIa genetic polymorphisms, factor VIIa, thrombin, endothelin-1, cardiac myofibrillar proteins, Fas and Fas ligand, ligands thereof or binding partners thereof or nucleic acid molecules encoding same or their fragments or ligands or binding partners and contacting said biological sample with a second set of members wherein one or more of said second set of members are binding partners to one or more of said first set of members and wherein the pattern of interaction between said first and second sets of members including the absence of interaction is indicative of said condition or event or a condition or event.

A further aspect of the present invention contemplates a method of treatment, said method comprising assessing the parameters associated with a condition or event associated with the systemic vasculature or assessing a risk of a condition or event occurring, said method comprising contacting a biological sample from a subject to be tested wherein said biological sample comprises one or more members which are present, absent, elevated or otherwise activated in a subject following said condition or event or a condition or event otherwise associated with an aberration wherein said members are selected from two or more of myoglobin, myosin light chain (MLC), myosin heavy chain (MHC), total creatine kinase (CK) including CK-MB, lactate dehydrogenase (LDH-H4), aspartate aminotransferase (AST), cardiac troponin I and T (cTn-I and cTn-T, respectively) and cTn-I and cTn-I RNA, fatty acid binding protein (FAB protein) including FABP1 and human heart-type, glycogen phosphorylase-BB isoenzyme, α-atrial natriuretic peptide (ANP), cytoplasmic FABP, brain natriuretic peptide (BNP), adrenomedullin (ADM), low density lipoprotein (LDL), very low density lipoprotein (VLDL), high density lipoprotein (HDL) and intermediate density lipoprotein (IDL), C reactive protein (CRP), serum amyloid A, P-selectin, prostaglandins, platelet-activating factor (PAF), histamine, tumor necrosis factor α (TNFα), soluble TNF receptor 2 (sTNFR2), fibrin, fibrinogen, fibrinolytic peptides, modified haemoglobin (HbA1c), ferritin, soluble intercellular adhesion molecule (ICAM) including soluble intercellular adhesion molecule-1 (ICAM1), heat shock proteins, apoB, apoA, apoE, homocysteine or parts thereof, Streptococcus sp., Porphyromonas gingivalis, Helicobacter pyrlori and Chlamydia pneumoniae or immunological relatives thereof, necrosis and platelet markers, leptin, vasopeptidase inhibitor of cardiac endogenous kinins, heparin, metalloproteinase-9, metalloproteinase-1 including its tissue inhibitor, angiotensin-converting enzyme, CD95/Apo1/Fas, hepatocyte growth factor, soluble vascular cell adhesion molecule-1 (VCAM1), plasma brain natriuretic peptide, angiotensin II type receptor, endothelial constitutive nitric oxide synthase, glycoprotein IIIa genetic polymorphisms, factor VIIa, thrombin, endothelin-1, cardiac myofibrillar proteins, Fas and Fas ligand, ligands thereof or binding partners thereof or nucleic acid molecules encoding same or their fragments or ligands or binding partners and contacting said biological sample with one or more antibodies or immunological equivalents thereof capable of binding to said one or more members in the biological sample and wherein the pattern of interaction between said members and antibodies including the absence of interaction is interaction between said members and antibodies including the absence of interaction is indicative of said condition or event and then effecting a suitable treatment regimen.

Yet another aspect of the present invention comprises an array of binding partners for members in a biological sample from a subject said members being present, absent, elevated or otherwise activated in a subject following a condition or event associated with the systemic vasculature wherein the binding partners are defined by (x,y) coordinates such that the array comprises n binding partners at coordinates (x,y), (x₂, y₂) . . . (xₙ,yₙ) and wherein the pattern of interaction between the members and the binding partners is indicative of said condition or event.

Still another aspect of the present invention provides a method for estimating the size of an infarct or related condition in a subject wherein the size of the infarct (Is) is determined by the formula:—

$$Is = \frac{{\int }_{0}^{t} f ( t ) ⅆ t \times Bw \times Kw}{Ed \times Kr}$$

wherein
Is is infarct size
F(t)dt is the rate of release of a member in a biological sample, said member being present, absent, elevated or otherwise activated in a subject following a cardiovascular condition or event leading to the infarct [f(t) is also known as the member appearance function];
Bw is the body weight of the subject;
Kw is the proportion of the body weight into which the member is released;
Ed is the rate of removal of the member from evaluation; and
Kr is the total amount of member released divided by the amount of the member released from the infarcted tissue;
   said method comprising contacting a biological sample from said subject wherein said sample comprises members present, absent, elevated or otherwise activated in a subject following a cardiovascular condition or event or a condition or event otherwise associated with a cardiovascular aberration with one or more binding partners of said members wherein the binding partners are immobilized to a solid support and wherein the pattern of interaction between the members and binding partners is indicative of the size of the infarct or otherwise provides data input for assessment of the size of the infarct.

Even still another aspect of the present invention contemplates a method of assessing the parameters associated with a condition or event associated with the systemic vascularization, said method comprising screening for the presence of two or more mRNA molecules in a biological sample which mRNA molecules are translatable to members which are present, absent, elevated or otherwise activated following a cardiovascular condition or event or a condition or event otherwise associated with a cardiovascular aberration said screening comprising contacting the biological sample with an array of oligonucleotides capable of hybridizing or otherwise capturing said mRNA molecule and detecting said hybridization or captive and wherein presence or absence of said mRNA molecule is indicative of a said condition or event or a risk of development of same.

Even yet another aspect of the present invention provides a method for estimating the risk of developing a condition or event of the systemic vasculature in a subject wherein the risk is a function of the presence or absence of one or more of myoglobin, myosin light chain (MLC), myosin heavy chain (MHC), total creatine kinase (CK) including CK-MB, lactate dehydrogenase (LDH-H4), aspartate aminotransferase (AST), cardiac troponin I and T (cTn-I and cTn-T, respectively) and cTn-I and cTn-I RNA, fatty acid binding protein (FAB protein) including FABP1 and human heart-type, glycogen phosphorylase-BB isoenzyme, α-atrial natriuretic peptide (ANP), cytoplasmic FABP, brain natriuretic peptide (BNP), adrenomedullin (ADM), low density lipoprotein (LDL), very low density lipoprotein (VLDL), high density lipoprotein (HDL) and intermediate density lipoprotein (IDL), C reactive protein (CRP), serum amyloid A, P-selectin, prostaglandins, platelet-activating factor (PAF), histamine, tumor necrosis factor α (TNFα), soluble TNF receptor 2 (sTNFR2), fibrin, fibrinogen, fibronolytic peptides, modified haemoglobin (HbA1c), adhesion molecule-1 (ICAM1), heat shock proteins, apoB, apoA, apoE, homocysteine or parts thereof, Streptococcus sp., Porphyromonas gingivalis, Helicobacter pylori and Chlamydia pneumoniae or immunological relatives thereof, necrosis and platelet markers, leptin, vasopeptidase inhibitor of cardiac endogenous kinins, heparin, metalloproteinase-9, metalloproteinase-1 including its tissue inhibitor, angiotensin-converting enzyme, CD95/Apo1/Fas, hepatocyte growth factor, soluble vascular cell adhesion molecule-1 (VCAM1), plasma brain natriuretic peptide, angiotensin II type receptor, endothelial constitutive nitric oxide synthase, glycoprotein IIIa genetic polymorphisms, factor VIIa, thrombin, endothelin-1, cardiac myofibrillar proteins, Fas and Fas ligand, ligands thereof or binding partners thereof or nucleic acid molecules encoding same or their fragments or ligands or binding partners.

Another aspect of the present invention contemplates a method of estimating the risk of developing ACS including AMI or a related condition in a subject said method comprising screening for the presence of two or more mRNA molecules in a biological sample which mRNA molecules are translatable to members which are present, absent, elevated or otherwise activated following a cardiovascular condition or event or a condition or event otherwise associated with a cardiovascular aberration said screening comprising contacting the biological sample with an array of oligonucleotides capable of hybridizing or otherwise capturing said mRNA molecule and detecting said hybridization or captive and wherein the presence or absence of said mRNA molecule is indicative of a cardiovascular condition or event or a condition or event otherwise associated with a cardiovascular aberration or a risk of development of same.

A further aspect of the invention is directed to a data processing means for assessing a condition or event of the systemic vasculature, said data processing means executing the steps of:—
(1) detecting a reporter molecule as an indicator of interaction or absence of interaction with an immobilized member on a biochip array;
(2) analyzing the data obtained in (1) to identify members present in a biological sample;
(3) optionally quantitating the amount of members from (2); and
(4) analyzing the data to attribute a likelihood or risk of a condition or event.

Yet another aspect of the present invention contemplates a computer program product for assessing the likelihood of or risk of development of a condition or event associated with the systemic vasculature, said product comprising:—
(1) code that receives an input value for one or more of features wherein said features are selected from:—
   (a) absence or presence of myoglobin;
   (b) absence or presence of myosin light chain (MLC);
   (c) absence or presence of myosin heavy chain (MHC);
   (d) absence or presence of total creatine kinase (CK) including CK-MB;
   (e) absence or presence of lactate dehydrogenase (LDH-H4);
   (f) absence or presence of aspartate aminotransferase (AST);
   (g) absence or presence of cardiac troponin I and T (cTn-I and cTn-T, respectively) and cTn-I and cTn-I RNA;
   (h) absence or presence of fatty acid binding protein (FAB protein) including FABP1 and human heart-type;
   (i) absence or presence of glycogen phosphorylase-BB isoenzyme;
   (j) absence or presence of α-atrial natriuretic peptide (ANP);
   (k) cytoplasmic FABP;
   (l) absence or presence of brain natriuretic peptide (BNP);
   (m) absence or presence of adrenomedullin (ADM);
   (n) absence or presence of low density lipoprotein (LDL), very low density density lipoprotein (IDL);
   (o) absence or presence of C reactive protein (CRP);
   (p) absence or presence of serum amyloid A;
   (q) absence or presence of P-selectin;
   (r) absence or presence of prostaglandins;
   (s) absence or presence of platelet-activating factor (PAF);
   (t) absence or presence of histamine;
   (u) absence or presence of tumor necrosis factor α (TNFα);
   (v) absence or presence of soluble TNF receptor 2 (sTNFR2);
   (w) absence or presence of fibrin;
   (x) absence or presence of fibrinogen;
   (y) absence or presence of fibrinolytic peptides;
   (z) absence or presence of modified haemoglobin (HbA1c);
   (aa) absence or presence of ferritin;
   (bb) absence or presence of soluble intercellular adhesion molecule (ICAM) including soluble intercellular adhesion molecule-1 (ICAM);
   (cc) absence or presence of heat shock proteins;
   (dd) absence or presence of apoB, apoA, apoE;
   (ee) absence or presence of homocysteine or parts thereof;
   (ff) absence or presence of Streptococcus sp., Porphyromonas gingivalis, Helicobacter pylori or Chlamydia pneumoniae or immunological relatives thereof;
   (gg) absence or presence of necrosis and platelet markers;
   (hh) absence or presence of leptin;
   (ii) absence or presence of vasopeptidase inhibitor of cardiac endogenous kinins;
   (jj) absence or presence of heparin;
   (kk) absence or presence of metalloproteinase-9;
   (ll) absence or presence of metalloproteinase-1 including its tissue inhibitor;
   (mm) absence or presence of angiotensin-converting enzyme;
   (nn) absence or presence of CD95/Apo1/Fas;
   (nn) absence or presence of CD95/Apo 1/Fas;
   (oo) absence or presence of hepatocyte growth factor;
   (pp) absence or presence of soluble vascular cell adhesion molecule-1 (VCAM1);
   (qq) absence or presence of plasma brain natriuretic peptide;
   (rr) absence or presence of angiotensin II type receptor;
   (ss) absence or presence of endothelial constitutive nitric oxide synthase;
   (tt) absence or presence of glycoprotein IIIa genetic polymorphisms;
   (uu) absence or presence of factor VIIa;
   (vv) absence or presence of thrombin;
   (ww) absence or presence of endothelin-1;
   (xx) absence or presence of cardiac myofibrillar proteins;
   (yy) absence or presence of Fas and Fas ligand; and
   (zz) absence or presence of ligands thereof or binding partners thereof or nucleic acid molecules encoding same or their fragments or ligands or binding partners; and
(2) a computer readable medium that stores the code.

Still another aspect of the invention extends to a computer system for assessing the likelihood of a subject having a condition or event associated with the systemic vascularization wherein said computer system comprises:—
(1) a machine-readable data storage medium comprising a data storage material encoded with machine-readable data, wherein said machine-readable data comprise values for one or more features, wherein said features are selected from:—
   (a) absence or presence of myoglobin;
   (b) absence or presence of myosin light chain (MLC);
   (c) absence or presence of myosin heavy chain (MHC);
   (d) absence or presence of total creatine kinase (CK) including CK-MB;
   (f) absence or presence of aspartate aminotransferase (AST);
   (g) absence or presence of cardiac troponin I and T (cTn-I and cTn-T, respectively) and cTn-I and cTn-I RNA;
   (h) absence or presence of fatty acid binding protein (FAB protein) including FABP1 and human heart-type;
   (i) absence or presence of glycogen phosphorylase-BB isoenzyme;
   (j) absence or presence of α-atrial natriuretic peptide (ANP);
   (k) cytoplasmic FABP;
   (l) absence or presence of brain natriuretic peptide (BNP);
   (m) absence or presence of adrenomedullin (ADM);
   (n) absence or presence of low density lipoprotein (LDL), very low density lipoprotein (VLDL), high density lipoprotein (HDL) and intermediate density lipoprotein (IDL);
   (o) absence or presence of C reactive protein (CRP);
   (p) absence or presence of serum amyloid A;
   (q) absence or presence of P-selectin;
   (r) absence or presence of prostaglandins;
   (s) absence or presence of platelet-activating factor (PAF);
   (t) absence or presence of histamine;
   (u) absence or presence of tumor necrosis factor α (TNFα);
   (v) absence or presence of soluble TNF receptor 2 (sTNFR2);
   (w) absence or presence of fibrin;
   (x) absence or presence of fibrinogen;
   (y) absence or presence of fibrinolytic peptides;
   (z) absence or presence of modified haemoglobin (HbA1c);
   (aa) absence or presence of ferritin;
   (bb) absence or presence of soluble intercellular adhesion molecule (ICAM) including soluble intercellular adhesion molecule-1 (ICAM1);
   (cc) absence or presence of heat shock proteins;
   (dd) absence or presence of apoB, apoA, apoE;
   (ee) absence or presence of homocysteine or parts thereof;
   (ee) absence or presence of homocysteine or parts thereof;
   (ff) absence or presence of Streptococcus sp., Porphyromonas gingivalis, Helicobacter pylori or Chlamydia pneumoniae or immunological relatives thereof;
   (gg) absence or presence of necrosis and platelet markers;
   (hh) absence or presence of leptin;
   (ii) absence or presence of vasopeptidase inhibitor of cardiac endogenous kinins;
   (jj) absence or presence of heparin;
   (kk) absence or presence of metalloproteinase-9;
   (ll) absence or presence of metalloproteinase-1 including its tissue inhibitor;
   (mm) absence or presence of angiotensin-converting enzyme;
   (nn) absence or presence of CD95/Apo1/Fas;
   (oo) absence or presence of hepatocyte growth factor;
   (pp) absence or presence of soluble vascular cell adhesion molecule-1 (VCAM1);
   (qq) absence or presence of plasma brain natriuretic peptide;
   (rr) absence or presence of angiotensin II type receptor;
   (ss) absence or presence of endothelial constitutive nitric oxide synthase;
   (tt) absence or presence of glycoprotein IIIa genetic polymorphisms;
   (uu) absence or presence of factor VIIa;
   (vv) absence or presence of thrombin;
   (ww) absence or presence of endothelin-1;
   (xx) absence or presence of cardiac myofibrillar proteins;
   (yy) absence or presence of Fas and Fas ligand; and
   (zz) absence or presence of ligands thereof or binding partners thereof or nucleic acid molecules encoding same or their fragments or ligands or binding partners;
(2) a working memory for storing instructions for processing said machine-readable data;
(3) a central-processing unit coupled to said working memory and to said machine-readable data storage medium, for processing said machine readable data to provide a sum of said values corresponding to a predictive value for said candidate sequences; and
(4) an output hardware coupled to said central processing unit for receiving said predictive value.

### BRIEF DESCRIPTION OF THE FIGURES

FIG. 1a is a diagrammatic representation showing the detection of a cardiac marker (CM) which has interacted with a binding partner (BP) immobilized to a solid support (SS). A reporter molecule (RM) is attached to the CM and the RM provides an identifiable signal (IS).

FIGS. 1b to 1e are diagrammatic representations showing various detection of interaction protocols between an immobilized member (an antibody; Ab) and its binding partner (cardiac marker; CM). In one aspect, (FIG. 1b) biotin ( [symbol] ) is used to label CM. A fusion protein consisting of streptavidin (SA) and alkaline phosphatase (AP) is used to attach to the biotin portion of the biotin-labeled CM. The AP converts a substrate (S) to a detectable product (P). The Ab, is immobilized to a solid substrate. In FIG. 1c, the captured CM is detected by a fluorescent (Fl) AP/HRP or radioactively (Rad) labeled anti-CM antibody. In FIG. 1d, soluble anti-CM antibody is used to bind to the immobilized CM and then itself detected by an anti-immunoglobulin antibody labeled with a fluorescent tag or other RMs. In FIG. 1e, the CM is labeled with a fluorescent member or other RM.

FIG. 2 is a graphical representation showing the time-courses for release of cardiac markers into the blood following onset of chest pain.

FIG. 3 is a diagrammatic representation of a system used to carry out the instructions to diagnose a particular condition or event of the systemic vasculature wherein the instructions are encoded by the storage medium.

FIG. 4 is a diagrammatic representation of a cross-section of a magnetic storage medium to diagnose a particular condition or event of the systemic vasculature.

FIG. 5 is a diagrammatic representation of a cross-section of an optically readable data storage system to diagnose a particular condition or event of the systemic vasculature.

### DETAILED DESCRIPTION OF THE PREFERRED EMBODIMENTS

The present invention is predicated in part on the development of an assay device which is capable of assessing two or more parameters associated with a condition or event associated with the systemic vasculature. The detection of parameters permits both predictive and diagnostic analyses to be undertaken. The term “predictive” in this context includes determining the risk factor for a healthy or unhealthy subject including a human of development of a condition or experiencing an event.

Accordingly, one aspect of the present invention contemplates a method of assessing the parameters associated with a condition or event of the systemic vasculature or assessing a risk of a condition or event occurring, said method comprising obtaining a biological sample from a subject to be tested wherein said biological sample comprises one or more members which are present, absent, elevated or otherwise activated or up or down regulated in a subject prior to, during or following said condition or event and contacting said biological sample with a second set of members wherein one or more of said second set of members are binding partners to one or more of said first set of members and wherein the pattern of interaction between said first and second sets of members including the absence of interaction is indicative of said condition or event or the risk of development of same.

A condition or event associated with the systemic vasculature includes a vascular disease including cardiovascular, stroke, pulmonary, renovascular, cerebrovascular, thrombotic or generalized arterial or venous condition or event, organ failure including liver, kidney or heart failure, tissue rejection such as organ transplant rejection, a thrombotic event including deep vein thrombosis, an infection, damage to vessels of the circulatory system, stent failure or trauma caused by a stent, pace-maker or other prosthetic device, vascularization of a tumor, events or conditions following surgery such as a hip replacement or knee reconstruction, trauma or age-related disease and endothelial damage. Reference herein to a “cardiovascular condition or event” includes heart diseases, heart attack, heart disorders and infections in the heart as well as heart failure. In a particularly preferred embodiment, the cardiovascular condition or event is acute coronary syndrome (ACS) or a related disorder such as but not limited to coronary arterial disease and reperfusion following therapeutic intervention such as balloon angioplasty or thrombolytic therapy. The term “ACS” includes acute myocardial infarction (AMI). A cardiovascular condition or event includes congestive heart failure which occurs when the heart is unable to produce sufficient output to meet the needs of the body's metabolism. There are four categories of such failure:—
Class I: patients with coronary artery disease (CAD) and other disorders where ordinary physical activity does not cause fatigue, palpitations, shortness of breath or anginal (heart) pain;
Class II: as in Class I but where patients are comfortable at rest but where there is slight limitation resulting from exercise;
Class III: as in Class II but where there is now a marked limitation to physical activity, and
Class IV: as in Class III but where any physical activity causes angina and discomfort.

All such conditions and events are encompassed by the term “cardiovascular condition or event” and are also encompassed by a condition or event associated with the systemic vasculature. A cardiovascular condition or event includes cardiovascular aberration such as caused by infections by microorganisms and viruses. Examples of microbial infections include those caused by or associated with infections by Chlamydia pneumoniae (lungs), Porphyromonas gingivalis (mouth), Streptococcus sanguis or Helicobacter pylori (gut). Examples of viral infections include those caused by or associated with Cytomegalovirus or Coxsackie virus.

The term “biological sample” is used in its broadest sense and includes blood, serum, saliva, tissue fluid, synovial fluid, lymph, tissue or tissue extract, heart tissue, mucus, cerebrospinal fluid, urine, semen, fecal sample or any other sample derived from the subject and which might contain one or more members which are present, absent, elevated or otherwise activated in a subject following a condition or event associated with the systemic vasculature. A biological sample may also contain infectious agents or products therefrom. A biological sample may be obtained at any time including when a subject or patient is away from a point of medical care, when attended to by emergency care operators, during triage and during surgery or other interventionist procedure amongst other times. A “biological sample” may also include a supply of blood, blood products or tissue such as at a blood bank or organ bank.

Reference to a “subject” or “patient” includes a human, primate, livestock animal (e.g. sheep, horses, pigs, cows, donkeys), laboratory test animal (e.g. mice, rats, rabbits, guinea pigs) and companion animals (e.g. dogs, cats). The present invention has, therefore, application in the human, medical, veterinary and livestock industries.

The “members” in the biological sample include enzymes (such as isoenzymes), peptides, polypeptides, proteins, antibodies, lipids and complexes containing lipids including lipoproteins and/or carbohydrates and complexes containing carbohydrates as well as nucleic acid molecules including RNA or DNA or fragments thereof and complexes containing nucleic acid molecules. A “member” is also referred to herein as a “marker”. The members may also be microorganisms or viruses or parts thereof or products therefrom. A “part” of a microorganism or virus includes cell wall, cell wall fragments or components, cell membrane, flagella, carbohydrate complexes and antigens. A “product” includes metabolic by-products and cytoplasmic and membrane associated components. The RNA or DNA may arise from cell necrosis or changes in levels of mRNA may be detected when expression of DNA is increased or decreased.

Members in the biological sample preferably have a significant or substantial presence or absence in heart tissue or in the tissue of other organs or have a significant distribution in other tissues and provide the earliest possible indication of myocardial injury; provide a medium term indication of an event or condition associated with the systemic vasculature such as a cardiovascular condition or event (e.g. myocardial injury).

An assay of the members should, therefore, provide an indication of the existence and/or seriousness of a condition or event or provide an indication of a silent condition or event. For example, the assay of the present invention permits the determination of the existence and size of an infarct or a silent ischemia or an unstable angina.

Preferably, the assay should be capable of operating at the point of care although, as stated above, the assay may be performed or samples collected at a number of different locations.

Particularly preferred members in the biological sample include myoglobin, myosin light chain (MLC), myosin heavy chain (MHC), total creatine kinase (CK) including CK-MB, lactate dehydrogenase (LDH-H4), aspartate aminotransferase (AST), cardiac troponin I and T (cTn-I and cTn-T, respectively) and cTn-I and cTn-I RNA, fatty acid binding protein (FAB protein) including FABP1 and human heart-type, glycogen phosphorylase-BB isoenzyme, α-atrial natriuretic peptide (ANP), cytoplasmic FABP, brain natriuretic peptide (BNP), adrenomedullin (ADM), low density lipoprotein (LDL), very low density lipoprotein (VLDL), high density lipoprotein (HDL) and intermediate density lipoprotein (IDL), C reactive protein (CRP), serum amyloid A, P-selectin, prostaglandins, platelet-activating factor (PAF), histamine, tumor necrosis factor α (TNFα), soluble TNF receptor 2 (sTNFR2), fibrin, fibrinogen, fibrinolytic peptides, modified haemoglobin (HbA1c), ferritin, soluble intercellular adhesion molecule (ICAM) including soluble intercellular adhesion molecule-1 (CAM1), heat shock proteins, apoB, apoA, apoE, homocysteine or parts thereof, Streptococcus sp., Porphyromonas gingivalis, Helicobacter pylori and Chlamydia pneumoniae or immunological relatives thereof, necrosis and platelet markers, leptin, vasopeptidase inhibitor of cardiac endogenous kinins, heparin, metalloproteinase-9, metalloproteinase-1 including its tissue inhibitor, angiotensin-converting enzyme, CD95/Apo1/Fas, hepatocyte growth factor, soluble vascular cell adhesion molecule-1 (VCAM1), plasma brain natriuretic peptide, angiotensin II type receptor, endothelial constitutive nitric oxide synthase, glycoprotein IIIa genetic polymorphisms, factor VIIa, thrombin, endothelin-1, cardiac myofibrillar proteins, Fas and Fas ligand, ligands thereof or binding partners thereof or nucleic acid molecules encoding same or their fragments or ligands or binding partners.

Reference to detecting Streptococcus sp., Porphyromonas gingivalis, Helicobacter pylori and Chlamydia pneumoniae includes in a preferred embodiment, detecting antigens or other cell-specific molecules from the organisms.

Although the above members are in the biological sample, they may also be included as part of the second set of members. In such a case, their binding partners are sought in a biological sample. In essence, one or more members in one set will have a binding partner in the other set of members.

The above-mentioned members may be either in the first set or in the second set. The second set of members is generally immobilized to a support such as but not limited to a solid support.

The solid support is typically glass or a polymer, such as but not limited to cellulose, ceramic material, nitrocellulose, polyacrylamide, nylon, polystyrene and its derivatives, polyvinylidene difluoride (PVDF), methacrylate and its derivatives, polyvinyl chloride or polypropylene. Nitrocellulose is particularly useful and preferred in accordance with the present invention. A solid support may also be a hybrid such as a nitrocellulose film supported on a glass or polymer matrix. Reference to a “hybrid” includes reference to a layered arrangement of two or more glass or polymer surfaces listed above. The solid support may be in the form of a membrane or tubes, beads, discs or microplates, or any other surface suitable for conducting an assay. Binding processes to immobilize the molecules are well-known in the art and generally consist of covalently binding (e.g. cross linking) or physically adsorbing the molecules to the solid substrate. Generally, solid supports are contacted with a blocking agent such as but not limited to skim milk, bovine serum albumin, human serum albumin, Irish moss extract or other sources of carrageenan or gelatin.

The expression “pattern of interaction” is used in its broadest context to include: the presence or absence of interaction relative to background interaction; the relative density of interaction such as the relative density of members bound to a binding partner on the solid support relative to background; the presence or absence of internal molecules in cells lysed on a discrete spot on the support; the relative number of members in a biological sample and/or; the differential expression of particular members. Any or all of the above criteria may be used to assess the interaction between an immobilized molecule and its binding partner. The pattern of expression or interaction may also be subject to quantitation. Reference to “presence” and “absence” includes substantial “presence” or “absence” as well as relative “presence” or “absence” compared to each other or any other marker.

Reference to “assessing the parameters” includes the determination of members within the biological sample which in turn is indicative of the presence of a cardiovascular condition or event associated with a systemic vasculature. The assessment becomes part of a risk analysis for the condition or event.

Accordingly, another aspect of the present invention contemplates a method of assessing the parameters associated with a condition or event associated with the systemic vasculature, said method comprising contacting a biological sample from a subject to be tested wherein said biological sample comprises one or more members which are present, absent, elevated or otherwise activated in a subject following said condition or event, said members being selected from two or more of myoglobin, myosin light chain (MIC), myosin heavy chain (MHC), total creatine kinase (CK) including CK-MB, lactate dehydrogenase (LDH-H4), aspartate aminotransferase (AST), cardiac troponin I and T (cTn-I and cTn-T, respectively) and cTn-I and cTn-I RNA, fatty acid binding protein (FAB protein) including FABP1 and human heart-type, glycogen phosphorylase-BB isoenzyme, α-atrial natriuretic peptide (ANP), cytoplasmic FABP, brain natriuretic peptide (BNP), adrenomedullin (ADM), low density lipoprotein (LDL), very low density lipoprotein (VLDL), high density lipoprotein (HDL) and intermediate density lipoprotein (IDL), C reactive protein (CRP), serum amyloid A, P-selectin, prostaglandins, platelet-activating factor (PAF), histamine, tumor necrosis factor α (TNFα), soluble TNF receptor 2 (sTNFR2), fibrin, fibrinogen, fibrinolytic peptides, modified haemoglobin (HbA1c), ferritin, soluble intercellular adhesion molecule (CAM) including soluble intercellular adhesion molecule-1 (ICAM1), heat shock proteins, apoB, apoA, apoE, homocysteine or parts thereof, Streptococcus sp., Porphyromonas gingivalis, Helicobacter pylori and Chlamydia pneumoniae or immunological relatives thereof, necrosis and platelet markers, leptin, vasopeptidase inhibitor of cardiac endogenous kinins, heparin, metalloproteinase-9, metalloproteinase-1 including its tissue inhibitor, angiotensin-converting enzyme, CD95/Apo1/Fas, hepatocyte growth factor, soluble vascular cell adhesion molecule-1 (VCAM1), plasma brain natriuretic peptide, angiotensin II type receptor, endothelial constitutive nitric oxide synthase, glycoprotein IIIa genetic polymorphisms, factor VIIa, thrombin, endothelin-1, cardiac myofibrillar proteins, Fas and Fas ligand, ligands thereof or binding partners thereof or nucleic acid molecules encoding same or their fragments or ligands or binding partners and contacting said biological sample with a second set of members wherein one or more of said second set of members are binding partners to one or more of said first set of members and wherein the pattern of interaction between said first and second sets of members including the absence of interaction is indicative of said condition or event or a condition or event.

In a particularly preferred embodiment, the second set of members immobilized to a solid support comprises antibodies which are potentially specific or generic for respective binding members in the biological sample. The latter includes immunointeractive molecules such as but not limited to analogues or antigenic fragments thereof or epitope containing fragments thereof.

In accordance with the present invention, the inventors have determined that the simultaneous identification of a range of markers associated with a cardiovascular condition or event associated with the systemic vasculature provides a more efficacious means of detecting the condition or event, estimating the time and size of the event when such an event is, for example, an infarction, detecting reperfusion and following interventional treatment such as balloon angioplasty or thrombolytic therapy as well as determining the risk factors associated with the conditions or events. The determination of more than one parameter simultaneously as opposed to singularly reduces the time of diagnosis and/or risk of misdiagnosis (e.g. improves the certainty of a particular diagnosis) and enables the more rapid implementation of a treatment protocol including administration of drugs, such as heart anti-clotting drugs (e.g. tissue plasminogen activator (tPA) or streptokinase) as well as surgical intervention or discharge from accident and emergency wards, centres or scenes including triage.

Accordingly, yet another aspect of the present invention contemplates a method of treatment, said method comprising assessing the parameters associated with a condition or event associated with the systemic vasculature or assessing a risk of a condition or event occurring, said method comprising contacting a biological sample from a subject to be tested wherein said biological sample comprises one or more members which are present, absent, elevated or otherwise activated in a subject following said condition or event or a condition or event otherwise associated with an aberration wherein said members are selected from two or more of myoglobin, myosin light chain (MLC), myosin heavy chain (MHC), total creatine kinase (CK) including CK-MB, lactate dehydrogenase (LDH-H4), aspartate aminotransferase (AST), cardiac troponin I and T (cTn-I and cTn-T, respectively) and cTn-I and cTn-I RNA, fatty acid binding protein (FAB protein) including FABP1 and human heart-type, glycogen phosphorylase-BB isoenzyme, α-atrial natriuretic peptide (ANP), cytoplasmic FABP, brain natriuretic peptide (BNP), adrenomedullin (ADM), low density lipoprotein (LDL), very low density lipoprotein (VLDL), high density lipoprotein (HDL) and intermediate density lipoprotein (IDL), C reactive protein (CRP), serum amyloid A, P-selectin, prostaglandins, platelet-activating factor (PAF), histamine, tumor necrosis factor α (TNFα), soluble TNF receptor 2 (sTNFR2), fibrin, fibrinogen, fibrinolytic peptides, modified haemoglobin (HbA1c), ferritin, soluble intercellular adhesion molecule (ICAM) including soluble intercellular adhesion molecule-1 (ICAM1), heat shock proteins, apoB, apoA, apoE, homocysteine or parts thereof, Streptococcus sp., Porphyromonas Porphyromonas gingivalis, Helicobacter pylori and Chlamydia pneumoniae or immunological relatives thereof, necrosis and platelet markers, leptin, vasopeptidase inhibitor of cardiac endogenous kinins, heparin, metalloproteinase-9, metalloproteinase-1 including its tissue inhibitor, angiotensin-converting enzyme, CD95/Apo1/Fas, hepatocyte growth factor, soluble vascular cell adhesion molecule-1 (VCAM1), plasma brain natriuretic peptide, angiotensin II type receptor, endothelial constitutive nitric oxide synthase, glycoprotein IIIa genetic polymorphisms, factor VIIa, thrombin, endothelin-1, cardiac myofibrillar proteins, Fas and Fas ligand, ligands thereof or binding partners thereof or nucleic acid molecules encoding same or their fragments or ligands or binding partners and contacting said biological sample with one or more antibodies or immunological equivalents thereof capable of binding to said one or more members in the biological sample and wherein the pattern of interaction between said members and antibodies including the absence of interaction is indicative of said condition or event and then effecting a suitable treatment regimen.

The present invention is particularly useful as a complement to treatments such as infusion of tPA (i.e. thrombolytic therapy), balloon angioplasty, stent insertion and/or coronary artery graft surgery (CAGS). The present invention is, therefore, an important adjunct to clinical practice.

Events and conditions associated with the systemic vasculature include inter alia a cardiovascular condition or event, trauma including following surgery, organ failure including heart, liver or kidney failure, stroke, thrombotic events including deep vein thrombosis, a pulmonary condition, a thrombotic event and vascularization of tumor or cancer tissue. The assay may be practiced to determine the risk of an otherwise healthy subject of developing a condition or event or an unhealthy person who is exposed to risk such as surgery or administration of drugs. Particularly important conditions and events contemplated by the present invention are cardiovascular conditions, pulmonary conditions and thrombotic events and risk assessments of developing same.

Particular markers may also be used for certain conditions. For example, stroke or brain injury or trauma markers include interleukins (e.g. IL-1, IL-6, IL-8, IL-10, IL-17) and TGFβ; neurological markers including amyloid β-1-42, amyloid β-1-40, tau, apoE, apoE4, S-100B, neuron-specific enolase and ubiquitin as well as or an alternative to the other markers listed above. Kidney trauma or disease may be identified by markers such as urinary glutathione S-transferase (GST), α-GST, creatine, melanin A, prostate specific antigen, citrate, acetate and erythropoietin. Other specific markers may also be employed, for example, lung or other pulmonary conditions.

The method of the present invention is conveniently practised using an array of immobilized binding partners to members in the biological sample. The term “array” is not to imply any limitation as to shape or order or pattern of the binding partners of the array and the binding partners may be arranged in a defined pattern or may be randomly or semi-randomly arranged. Generally, the array comprises two or more binding partners but more preferably comprises from about 2 to about 10,000, more preferably from about 10 to about 5000 and even more preferably from about 20 to about 1000. Preferably, the spots of the array are arranged in a sequence such as a rectangular, triangular or spherical matrix where the position of an immunoglobulin region is defined by coordinates based on, for example, row and column. The array of immunoglobulins may cover any convenient region such as from about 0.1 mm² to about 100 mm², preferably about 0.5 mm² to about 15 mm². Generally, each region or spot is made up of immunoglobulin(s) having a single distinct specificity. Specificity in this context is with respect to different antigens or different regions of the one antigen. The preferred number of immunoglobulin spots is from about 7 to about 1000 and more preferably from about 10 to about 1000. Most preferably, the immunoglobulins are arranged in multiples such as duplicates, triplicates or greater. Preferably, the array comprises the binding partners defined by partner (x,y) coordinates such that each binding partner is defined by coordinates (x,y), (x₂,y₂) . . . (xₙ,yₙ) wherein n is the number of binding partners for members in a biological sample. A pattern of interaction is obtained using two or more interactions.

Accordingly, another aspect of the present invention comprises an array of binding partners for members in a biological sample from a subject said members being present, absent, elevated or otherwise activated in a subject following a condition or event associated with the systemic vasculature wherein the binding partners are defined by (x,y) coordinates such that the array comprises n binding partners at coordinates (x,y), (x₂, y₂) . . . (xₙ,yₙ) and wherein the pattern of interaction between the members and the binding partners is indicative of said condition or event. The subscripts “1”, “2” and “n” in the above coordinates are not to imply that the x and y coordinate values are the same.

In one useful embodiment, the instant invention detects myocardial tissue damage such as occurs in ACS including AMI. The present invention is also useful for determining the infarct size. Knowledge of the existence of real cardiac damage facilitates early and rapid therapeutic intervention.

Accordingly, another aspect of the present invention provides a method for estimating the size of an infarct or related condition in a subject wherein the size of the infarct (Is) is determined by the formula:—

$$Is = \frac{{\int }_{0}^{t} f ( t ) ⅆ t \times Bw \times Kw}{Ed \times Kr}$$

wherein
Is is infarct size
F(t)dt is the rate of release of a member in a biological sample, said member being present, absent, elevated or otherwise activated in a subject following a cardiovascular condition or event leading to the infarct [f(t) is also known as the member appearance function];
Bw is the body weight of the subject;
Kw is the proportion of the body weight into which the member is released;
Ed is the rate of removal of the member from evaluation; and
Kr is the total amount of member released divided by the amount of the member released from the infarcted tissue.
   said method comprising contacting a biological sample from said subject wherein said sample comprises members present, absent, elevated or otherwise activated in a subject following a cardiovascular condition or event or a condition or event otherwise associated with a cardiovascular aberration with one or more binding partners of said members wherein the binding partners are immobilized to a solid support and wherein the pattern of interaction between the members and binding partners is indicative of the size of the infarct or otherwise provides data input for assessment of the size of the infarct.

The above equation may be simplified by combining constants to form Kd and defining

$$f ( t ) as \frac{ⅆ E}{ⅆ t} + KdE .$$

Accordingly, the equation becomes

$$Kd {\int }_{0}^{t} E ( t ) ⅆ t + E ( T )$$

where E(T) is the activity or level of the member when the results of the parameters have been referred to base line conditions.

Preferably, the members in the biological sample comprise one or more of myoglobin, myosin light chain (MLC), myosin heavy chain (MHC), total creatine kinase (CK) including CK-MB, lactate dehydrogenase (LDH-H4), aspartate aminotransferase (AST), cardiac troponin I and T (cTn-I and cTn-T, respectively) and cTn-I and cTn-I RNA, fatty acid binding protein (FAB protein) including FABP1 and human heart-type, glycogen phosphorylase-BB isoenzyme, α-atrial natriuretic peptide (ANP), cytoplasmic FABP, brain natriuretic peptide (BNP), adrenomedullin (ADM), low density lipoprotein (LDL), very low density lipoprotein (VLDL), high density lipoprotein (HDL) and intermediate density lipoprotein (IDL), C reactive protein (CRP), serum amyloid A, P-selectin, prostaglandins, platelet-activating factor (PAF), histamine, tumor necrosis factor α (TNFα), soluble TNF receptor 2 (sTNFR2), fibrin, fibrinogen, fibrinolytic peptides, modified haemoglobin (HbA1c), ferritin, soluble intercellular adhesion molecule (ICAM) including soluble intercellular adhesion molecule-1 (ICAM1), heat shock proteins, apoB, apoA, apoE, homocysteine or parts thereof, Streptococcus sp., Porphyromonas gingivalis, Helicobacter pylori and Chlamydia pneumoniae or immunological relatives thereof, necrosis and platelet markers, leptin, vasopeptidase inhibitor of cardiac endogenous kinins, heparin, metalloproteinase-9, metalloproteinase-1 including its tissue inhibitor, angiotensin-converting enzyme, CD95/Apo1/Fas, hepatocyte growth factor, soluble vascular cell adhesion molecule-1 (VCAM1), plasma brain natriuretic peptide, angiotensin II type receptor, endothelial constitutive nitric oxide synthase, glycoprotein IIIa genetic polymorphisms, factor VIIa, thrombin, endothelin-1, cardiac myofibrillar proteins, Fas and Fas ligand, ligands thereof or binding partners thereof or nucleic acid molecules encoding same or their fragments or ligands or binding partners thereof or nucleic acid molecules encoding same or their fragments or ligands or binding partners.

Even one time point is useful to detect the presence of myocardial damage where multiple cardiac markers are quantitated and the ratios of their plasma levels are calculated. However, multiple (e.g. two or more) time points are preferred. Multiple time points taken over seconds, minutes, hours, days, weeks and months might provide further information, for example, the time of onset, extent of damage and/or the efficacy of therapeutic measures. Preferably, from about two to about ten time points are selected. The assay is useful for determining the relative amounts or quantitative amounts or qualitative amounts of preferably two or more members over time. A series of curves are produced and the area under the curve is substantially proportional to the infarct size. An extrapolation back to time zero permits determination of the approximate time of the infarct.

Similar issues arise in the detection of other conditions or events associated with the systemic vasculature including thrombosis including deep vein thrombosis, endothelial damage, vascularization of de novo tumor or cancer tissue, pulmonary conditions and conditions of the arteries or veins.

As indicated above, the assay of the present invention may be conducted by analyzing nucleic acid molecules in the biological sample. In one aspect, total nucleic acids (e.g. including mRNA, RNA and DNA) are detected in the serum or other tissue fluid resulting from cell necrosis. In another aspect, mRNA levels are detected following increased or transcription and/or translation of nucleotide sequences to produce mRNA and amino acid sequences respectively. In any event, levels of nucleic acid molecules may be altered following a cardiovascular condition or event.

Accordingly, another aspect of the present invention contemplates a method of assessing the parameters associated with a condition or event associated with the systemic vasculature, said method comprising screening for the presence of two or more mRNA molecules in a biological sample which mRNA molecules are translatable to members which are present, absent, elevated or otherwise activated following said condition or event or a condition or event otherwise associated with a cardiovascular aberration said screening comprising contacting the biological sample with an array of oligonucleotides capable of hybridizing or otherwise capturing said mRNA or a cDNA corresponding to said mRNA molecule and detecting said hybridization or captive and wherein the presence or absence of said mRNA or cDNA molecule is indicative of a said condition or event or a risk of development of same.

Preferably, mRNA molecules are first subjected to reverse transcriptase to form complementary (or copy) DNA (cDNA). Reverse transcription polymerase chain reactions (RT-PCR) are particularly useful and are contemplated by the instant invention.

Real-time PCR is also useful to study the changing levels of expression of genetic sequences over time. Real-time PCR using the TaqMan (registered trade mark) system developed by PE Biosystems (Foster City, Calif., USA) allows rapid detection and quantitation of DNA without the need for labour intensive post-PCR processing such as gel electrophoresis and radioactive hybridization (Heid et al., 1996). In addition, the built-in 96-well format greatly increases the number of samples which can be simultaneously analyzed. The method uses the 5′ exonuclease activity of a Taq polymerase (AmpliTaq Gold, PE Biosystems, Foster City, Calif., USA) during primer extension to cleave a dual-labelled, fluorogenic probe hybridized to the target DNA between the PCR primers. Prior to cleavage, a reporter dye, such as 6-carboxyfluorescein (6-FAM) at the 5′ end of the probe is quenched by 6-carboxy-tetramethylrhodamine (TAMRA) through fluorescent resonance energy transfer. Following digestion, FAM is released. The resulting fluorescence is continuously measured in real-time at 518 nm during the log phase of product accumulation and is proportional to the number of copies of the target sequence.

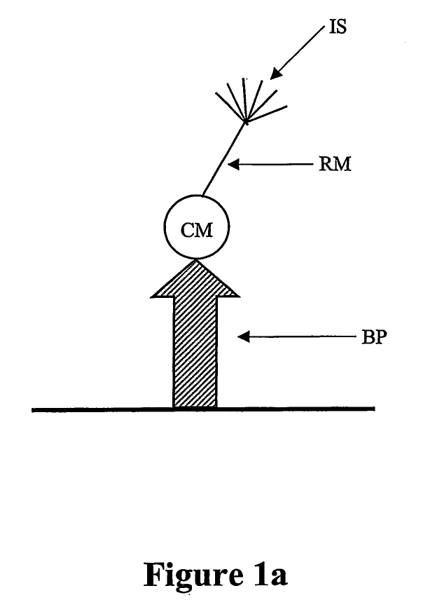

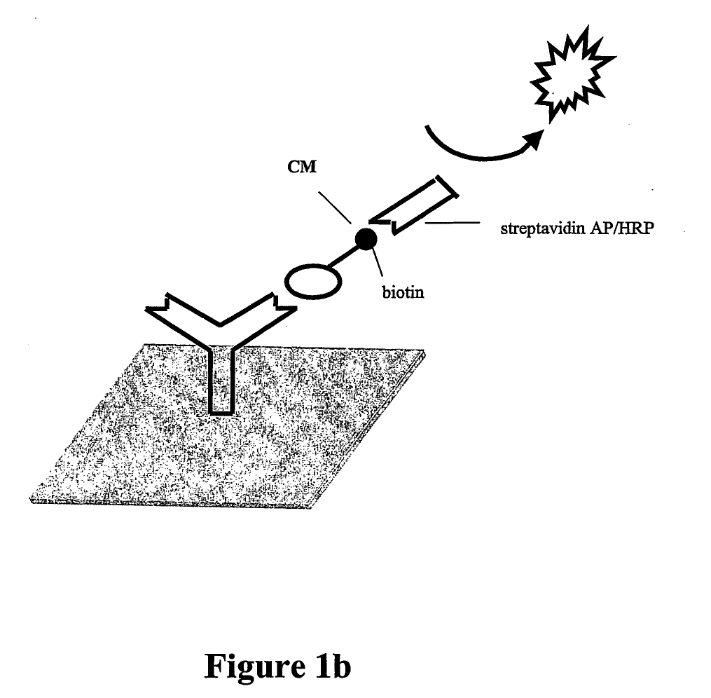

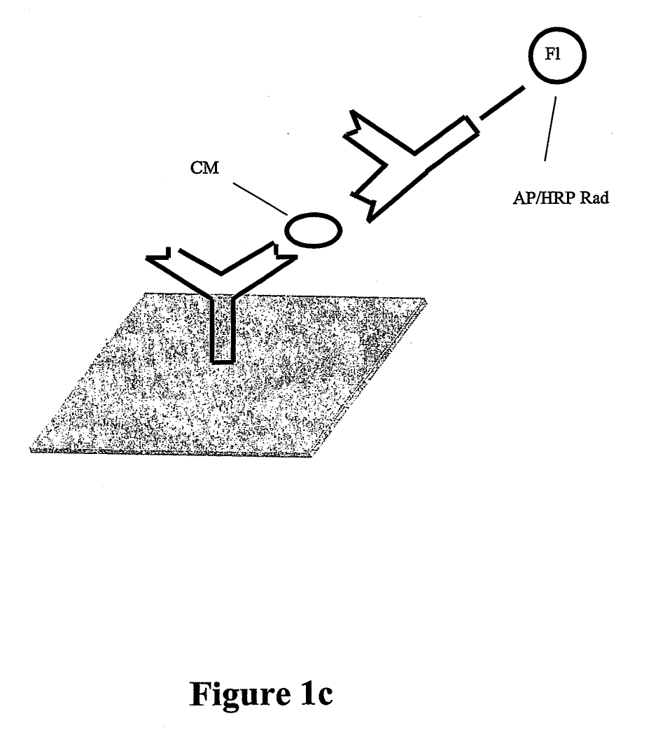

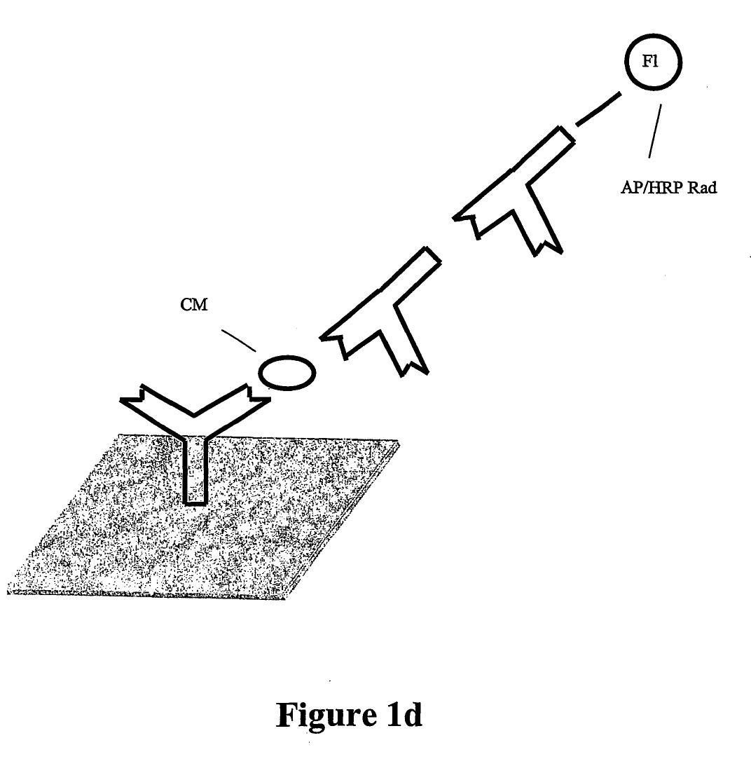

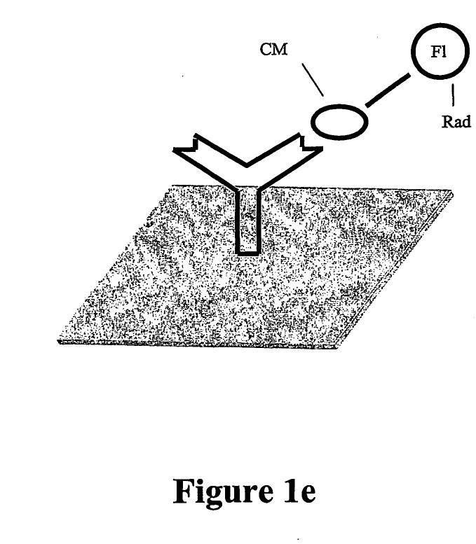

The detection of interaction between members and binding partners is conveniently accomplished using a reporter molecule. For example, the assay device may comprise an array of immunoglobulins. The immobilized array of immunoglobulins is then contacted by a biological sample. The contact is for a time and under conditions sufficient for antigens to be captured by the immobilized immunoglobulin. The captured antigens may then be detected by any convenient means such as biochemically, histochemically, immunologically or microscopically. Immunologic detection is particularly convenient. For example, a second immunoglobulin specific for a captured antigen, labelled with a reporter molecule, may be added. The identification of the reporter molecule indicates that the antigen is captured. Alternatively, after the second immunoglobulin is added and it forms a complex with the captured antigen, an anti-immunoglobulin labelled with a reporter molecule is added and the presence of a signal from the reporter molecule determined. A generalized method is shown in FIG. 1a. More specific and preferred methods are shown in FIGS. 1b-1e.

By “reporter molecule”, as used in the present specification, is meant a molecule which, by its chemical nature, provides an analytically identifiable signal which allows the detection of immunoglobulin bound antigen. Detection may be either qualitative or quantitative. The most commonly used reporter molecules in this type of assay include enzymes such as enzymes combined with light emitting molecules, fluorophores or radionuclide containing molecules (i.e. radioisotopes). In the case of an enzyme immunoassay, an enzyme is conjugated to the second or third immunoglobulin, generally by means of bifunctional cross-linking using, for example, agents such as glutaraldehyde, succinimide derivatives and the like. As will be readily recognized, however, a wide variety of different conjugation techniques exist which are readily available to one skilled in the art. Commonly used enzymes include horseradish peroxidase, glucose oxidase, β-galactosidase and alkaline phosphatase, amongst others. The substrates to be used with specific enzymes are generally chosen for the production, upon hydrolysis by the corresponding enzyme, of a detectable colour change. It is also possible to employ fluorogenic substrates which yield a fluorescent product or other photonic signal such as a flash of light.

Alternatively, fluorescent compounds, such as fluorescein and rhodamine, may be chemically coupled to immunoglobulins without altering their binding capacity. When activated by illumination with light of a particular wavelength, the fluorochrome-labelled immunoglobulin adsorbs the light energy, inducing a state of excitability in the molecule, followed by emission of the light at a characteristic colour visually detectable microscopically or using other imaging devices such as a confocal microscope or 2 dimensional laser scanner (e.g. FluorImager or Typhoon, Molecular Dynamics, Inc., Sunnyvale, USA).

In one particularly useful method, an immobilized immunoglobulin captures a member. A second antibody directed to a different or overlapping epitope is then immunointeracted to form an antibody-member-antibody complex. The second antibody is linked to, for example, streptavidin and alkaline phosphatase which permits production of an identifiable signal.

The assay device and the methods for conducting the assays of the present invention are readily adapted for automation. For example, robotic systems may be used to deliver appropriate amounts including nanolitre or picolitre volumes of immunoglobulins to a solid support such as a nitrocellulose film or microtitre plate. After appropriate treatment, the immobilized immunoglobulins may then be used in any assay. Again, this may be automated or conducted using robotics.

The present invention further contemplates the use of a binding partner array as herein described in the manufacture of an assay device for the detection of a cardiovascular condition or event or a condition associated with systemic vasculature.

The array of the present invention may also be adapted for use on a microchip. Microchip technology permits the generation of thousands of binding partners for a range of conditions and further permits automation and/or computer analysis. A “microchip” includes a matrix support comprising an array of adapter molecules, ligands or potential binding partners.

The matrix support may also be referred to as a biochip. Reference to a “biochip” also includes a “gene chip”. A gene chip includes any array of two or more oligonucleotides or polynucleotides immobilized to a solid support. The oligonucleotide or polynucleotide corresponds to a gene or mRNA sequence encoding a particular cardiac marker. Real-time PCR is one mechanism to screen for the rise and/or fall of markers. RT-PCR may also be used to detect the presence or absence of target nucleotide sequences by reading mRNA sequences back to corresponding cDNA sequences. Real-time PCR and in particular real-time-RT-PCR are particularly useful in determining changing patterns of expression of the markers.

The present invention further contemplates a data processing means to analyze and/or screen interaction between members and their binding partners. The data processing means preferably comprises a suitably programmed computer and the steps of the method are preferably performed using the suitably programmed computer. In various forms of the invention, the input information may take the form of values, identifiers or other data in respect of the identity of the interacting partners or the absence of interaction. The input data may be digitized. Alternatively, for implementation of the invention, a dedicated Fast Fourier transform chip can be employed as at least part of the processing means.

In a preferred form of the invention, representation measurements are made identifying or valuing the presence of an interaction between a member and a binding partner.

Accordingly, another aspect of the invention is directed to a data processing means for assessing a condition or event of the systemic vasculature, said data processing means executing the steps of:—
(1) detecting a reporter molecule as an indicator or interaction of absence of interaction with an immobilized member on a biochip array;
(2) analyzing the data obtained in (1) to identify members present in a biological sample;
(3) optionally quantitating the amount of members from (2); and
(4) analyzing the data to attribute a likelihood or risk of a condition or event.

In a particularly useful embodiment, the instant invention provides an indication of the likelihood of developing ACS such as AMI.

Accordingly, another useful aspect of the present invention provides a method for estimating the risk of developing a condition or event of the systemic vasculature in a subject wherein the risk is a function of the presence or absence of one or more of myoglobin, myosin light chain (MLC), myosin heavy chain (MHC), total creatine kinase (CK) including CK-MB, lactate dehydrogenase (LDH-H4), aspartate aminotransferase (AST), cardiac troponin I and T (cTn-I and cTn-T, respectively) and cTn-I and cTn-I RNA, fatty acid binding protein (FAB protein) including FABP1 and human heart-type, glycogen phosphorylase-BB isoenzyme, α-atrial natriuretic peptide (ANP), cytoplasmic FABP, brain natriuretic peptide (BNP), adrenomedullin (ADM), low density lipoprotein (LDL), very low density lipoprotein (VLDL), high density lipoprotein (HDL) and intermediate density lipoprotein (IDL), C reactive protein (CRP), serum amyloid A, P-selectin, prostaglandins, platelet-activating factor (PAF), histamine, tumor necrosis factor α (TNFα), soluble TNF receptor 2 (sTNFR2), fibrin, fibrinogen, fibrinolytic peptides, modified haemoglobin (HbA1c), ferritin, soluble intercellular adhesion molecule (ICAM) including soluble intercellular adhesion molecule-1 (ICAM1), heat shock proteins, apoB, apoA, apoE, homocysteine or parts thereof, Streptococcus sp., Porphyromonas gingivalis, Helicobacter pylori and Chlamydia pneumoniae or immunological relatives thereof, necrosis and platelet markers, leptin, vasopeptidase inhibitor of cardiac endogenous kinins, heparin, metalloproteinase-9, metalloproteinase-1 including its tissue inhibitor, angiotensin-converting enzyme, CD95/Apo1/Fas, hepatocyte growth factor, soluble vascular cell adhesion molecule-1 (VCAM1), plasma brain natriuretic peptide, angiotensin II type receptor, endothelial constitutive nitric oxide synthase, glycoprotein IIIa genetic polymorphisms, factor VIIa, thrombin, endothelin-1, cardiac myofibrillar proteins, Fas and Fas ligand, ligands thereof or binding partners thereof or nucleic acid molecules encoding same or their fragments or ligands or binding partners.

In a particularly useful embodiment, the present invention contemplates a method of estimating the risk of developing ACS including AMI or a related condition in a subject said method comprising screening for the presence of two or more mRNA molecules in a biological sample which mRNA molecules are translatable to members which are present, absent, elevated or otherwise activated following a cardiovascular condition or event or a condition or event otherwise associated with a cardiovascular aberration said screening comprising contacting the biological sample with an array of oligonucleotides capable of hybridizing or otherwise capturing said mRNA or its corresponding cDNA molecule and detecting said hybridization or captive and wherein the presence or absence of said mRNA or cDNA molecule is indicative of a cardiovascular condition or event or a condition or event otherwise associated with a cardiovascular aberration or a risk of development of same.

The determination of risk of cardiac aberration or other condition or event associated with the systemic vasculature has commercial benefits when an absence of risk is identified. For example, a hospital or emergency bed or emergency vehicle may not need to be employed. It is also useful to examine the general state of health or otherwise of a subject. Computer screening is particularly useful in determining risk analysis.

Thus, in another aspect, the invention contemplates a computer program product for assessing the likelihood of or risk of development of a condition or event associated with the systemic vasculature, said product comprising:—
(3) code that receives an input value for one or more of features wherein said features are selected from:—
   (a) absence or presence of myoglobin;
   (b) absence or presence of myosin light chain (MLC);
   (c) absence or presence of myosin heavy chain (MHC);
   (d) absence or presence of total creatine kinase (CK) including CK-MB;
   (e) absence or presence of lactate dehydrogenase (LDH-H4);
   (f) absence or presence of aspartate aminotransferase (AST);
   (g) absence or presence of cardiac troponin I and T (cTn-I and cTn-T, respectively) and cTn-I and cTn-I RNA;
   (h) absence or presence of fatty acid binding protein (FAB protein) including FABP 1 and human heart-type;
   (i) absence or presence of glycogen phosphorylase-BB isoenzyme;
   (j) absence or presence of α-atrial natriuretic peptide (ANP);
   (k) cytoplasmic FABP;
   (l) absence or presence of brain natriuretic peptide (BNP);
   (m) absence or presence of adrenomedullin (ADM);
   (n) absence or presence of low density lipoprotein (LDL), very low density lipoprotein (VLDL), high density lipoprotein (HDL) and intermediate density lipoprotein (IDL);
   (o) absence or presence of C reactive protein (CRP);
   (p) absence or presence of serum amyloid A;
   (q) absence or presence of P-selectin;
   (r) absence or presence of prostaglandins;
   (s) absence or presence of platelet-activating factor (PAF);
   (t) absence or presence of histamine;
   (u) absence or presence of tumor necrosis factor α (TNFα);
   (v) absence or presence of soluble TNF receptor 2 (sTNFR2);
   (w) absence or presence of fibrin;
   (x) absence or presence of fibrinogen;
   (y) absence or presence of fibrinolytic peptides;
   (z) absence or presence of modified haemoglobin (HbA1c);
   (aa) absence or presence of ferritin;
   (bb) absence or presence of soluble intercellular adhesion molecule (ICAM) including soluble intercellular adhesion molecule-1 (ICAM1);
   (cc) absence or presence of heat shock proteins;
   (dd) absence or presence of apoB, apoA, apoE;
   (ee) absence or presence of homocysteine or parts thereof;
   (ff) absence or presence of Streptococcus sp., Porphyromonas gingivalis, Helicobacter pylori or Chlamydia pneumoniae or immunological relatives thereof;
   (gg) absence or presence of necrosis and platelet markers;
   (hh) absence or presence of leptin;
   (ii) absence or presence of vasopeptidase inhibitor of cardiac endogenous kinins;
   (jj) absence or presence of heparin;
   (kk) absence or presence of metalloproteinase-9;
   (ll) absence or presence of metalloproteinase-1 including its tissue inhibitor;
   (mm) absence or presence of angiotensin-converting enzyme;
   (nn) absence or presence of CD95/Apo1/Fas;
   (oo) absence or presence of hepatocyte growth factor;
   (pp) absence or presence of soluble vascular cell adhesion molecule-1 (VCAM1);
   (qq) absence or presence of plasma brain natriuretic peptide;
   (rr) absence or presence of angiotensin II type receptor;
   (ss) absence or presence of endothelial constitutive nitric oxide synthase;
   (tt) absence or presence of glycoprotein IIIa genetic polymorphisms;
   (uu) absence or presence of factor VIIa;
   (vv) absence or presence of thrombin;
   (ww) absence or presence of endothelin-1;
   (xx) absence or presence of cardiac myofibrillar proteins;
   (yy) absence or presence of Fas and Fas ligand; and
   (zz) absence or presence of ligands thereof or binding partners thereof or nucleic acid molecules encoding same or their fragments or ligands or binding partners; and
(4) a computer readable medium that stores the code.

Yet another aspect of the invention extends to a computer system for assessing the likelihood of a subject having a condition or event associated with the systemic vasculaure wherein said computer system comprises:—
(1) a machine-readable data storage medium comprising a data storage material encoded with machine-readable data, wherein said machine-readable data comprise values for one or more features, wherein said features are selected from:—
   (a) absence or presence of myoglobin;
   (b) absence or presence of myosin light chain (MLC);
   (c) absence or presence of myosin heavy chain (MHC);
   (d) absence or presence of total creatine kinase (CK) including CK-MB;
   (e) absence or presence of lactate dehydrogenase (LDH-H4);
   (f) absence or presence of aspartate aminotransferase (AST);
   (g) absence or presence of cardiac troponin I and T (cTn-I and cTn-T, respectively) and cTn-I and cTn-I RNA;
   (h) absence or presence of fatty acid binding protein (FAB protein) including FABP1 and human heart-type;
   (i) absence or presence of glycogen phosphorylase-BB isoenzyme;
   (j) absence or presence of α-atrial natriuretic peptide (ANP);
   (k) cytoplasmic FABP;
   (l) absence or presence of brain natriuretic peptide (BNP);
   (m) absence or presence of adrenomedullin (ADM);
   (n) absence or presence of low density lipoprotein (LDL), very low density lipoprotein (VLDL), high density lipoprotein (HDL) and intermediate density lipoprotein (IDL);
   (o) absence or presence of C reactive protein (CRP);
   (p) absence or presence of serum amyloid A;
   (q) absence or presence of P-selectin;
   (r) absence or presence of prostaglandins;
   (s) absence or presence of platelet-activating factor (PAF);
   (t) absence or presence of histamine;
   (u) absence or presence of tumor necrosis factor α (TNFα);
   (v) absence or presence of soluble TNF receptor 2 (sTNFR2);
   (w) absence or presence of fibrin;
   (x) absence or presence of fibrinogen;
   (y) absence or presence of fibrinolytic peptides;
   (z) absence or presence of modified haemoglobin (HbA1c);
   (aa) absence or presence of ferritin;
   (bb) absence or presence of soluble intercellular adhesion molecule (ICAM) including soluble intercellular adhesion molecule-1 (ICAM1);
   (cc) absence or presence of heat shock proteins;
   (dd) absence or presence of apoB, apoA, apoE;
   (ee) absence or presence of homocysteine or parts thereof;
   (ff) absence or presence of Streptococcus sp., Porphyromonas gingivalis, Helicobacter pylori or Chlamydia pneumoniae or immunological relatives thereof;
   (gg) absence or presence of necrosis and platelet markers;
   (hh) absence or presence of leptin;
   (ii) absence or presence of vasopeptidase inhibitor of cardiac endogenous kinins;
   (jj) absence or presence of heparin;
   (kk) absence or presence of metalloproteinase-9;
   (ll) absence or presence of metalloproteinase-1 including its tissue inhibitor;
   (mm) absence or presence of angiotensin-converting enzyme;
   (nn) absence or presence of CD95/Apo1/Fas;
   (oo) absence or presence of hepatocyte growth factor;
   (pp) absence or presence of soluble vascular cell adhesion molecule-1 (VCAM1);
   (qq) absence or presence of plasma brain natriuretic peptide;
   (rr) absence or presence of angiotensin II type receptor;
   (ss) absence or presence of endothelial constitutive nitric oxide synthase;
   (tt) absence or presence of glycoprotein IIIa genetic polymorphisms;
   (uu) absence or presence of factor VIIa;
   (vv) absence or presence of thrombin;
   (ww) absence or presence of endothelin-1;
   (xx) absence or presence of cardiac myofibrillar proteins;
   (yy) absence or presence of Fas and Fas ligand; and
   (zz) absence or presence of ligands thereof or binding partners thereof or nucleic acid molecules encoding same or their fragments or ligands or binding partners;
(2) a working memory for storing instructions for processing said machine-readable data;
(3) a central-processing unit coupled to said working memory and to said machine-readable data storage medium, for processing said machine readable data to provide a sum of said values corresponding to a predictive value for said candidate sequences; and
(4) an output hardware coupled to said central processing unit for receiving said predictive value.

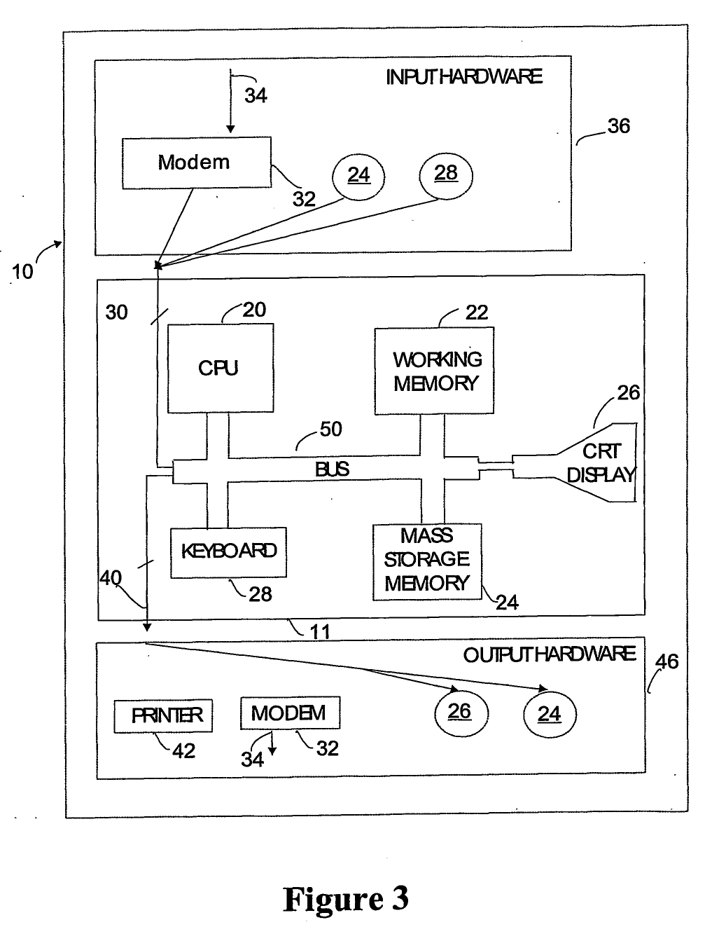

A version of these embodiments is presented in FIG. 3, which shows a system 10 including a computer 11 comprising a central processing unit (“CPU”) 20, a working memory 22 which may be, e.g. RAM (random-access memory) or “core” memory, mass storage memory 24 (such as one or more disk drives or CD-ROM drives), one or more cathode-ray tube (“CRT”) display terminals 26, one or more keyboards 28, one or more input lines 30, and one or more output lines 40, all of which are interconnected by a conventional bidirectional system bus 50.

Input hardware 36, coupled to computer 11 by input lines 30, may be implemented in a variety of ways. For example, machine-readable data of this invention may be inputted via the use of a modem or modems 32 connected by a telephone line or dedicated data line 34. Alternatively or additionally, the input hardware 36 may comprise a CD. Alternatively, ROM drives or disk drives 24 in conjunction with display terminal 26, keyboard 28 may also be used as an input device.

Output hardware 46, coupled to computer 11 by output lines 40, may similarly be implemented by conventional devices. By way of example, output hardware 46 may include CRT display terminal 26 for displaying a synthetic polynucleotide sequence or a synthetic polypeptide sequence as described herein. Output hardware might also include a printer 42, so that hard copy output may be produced, or a disk drive 24, to store system output for later use.

In operation, CPU 20 coordinates the use of the various input and output devices 36, 46 coordinates data accesses from mass storage 24 and accesses to and from working memory 22, and determines the sequence of data processing steps. A number of programs may be used to process the machine readable data of this invention. Exemplary programs may use for example the following steps:—
(1) inputting values for at least one feature associated with an expression of a target gene, wherein said features are selected from:—
   (a) absence or presence of myoglobin;
   (b) absence or presence of myosin light chain (MLC);
   (c) absence or presence of myosin heavy chain (MHC);
   (d) absence or presence of total creatine kinase (CK) including CK-MB;
   (e) absence or presence of lactate dehydrogenase (LDH-H4);
   (f) absence or presence of aspartate aminotransferase (AST);
   (g) absence or presence of cardiac troponin I and T (cTn-I and cTn-T, respectively) and cTn-I and cTn-I RNA;
   (h) absence or presence of fatty acid binding protein (FAB protein) including FABP1 and human heart-type;
   (i) absence or presence of glycogen phosphorylase-BB isoenzyme;
   (j) absence or presence of α-atrial natriuretic peptide (ANP);
   (k) cytoplasmic FABP;
   (l) absence or presence of brain natriuretic peptide (BNP);
   (m) absence or presence of adrenomedullin (ADM);
   (n) absence or presence of low density lipoprotein (LDL), very low density lipoprotein (VLDL), high density lipoprotein (HDL) and intermediate density lipoprotein (IDL);
   (o) absence or presence of C reactive protein (CRP);
   (p) absence or presence of serum amyloid A;
   (q) absence or presence of P-selectin;
   (r) absence or presence of prostaglandins;
   (s) absence or presence of platelet-activating factor (PAF);
   (t) absence or presence of histamine;
   (u) absence or presence of tumor necrosis factor α (TNFα);
   (v) absence or presence of soluble TNF receptor 2 (sTNFR2);
   (w) absence or presence of fibrin;
   (x) absence or presence of fibrinogen;
   (y) absence or presence of fibrinolytic peptides;
   (z) absence or presence of modified haemoglobin (HbA1c);
   (aa) absence or presence of ferritin;
   (bb) absence or presence of soluble intercellular adhesion molecule (ICAM) including soluble intercellular adhesion molecule-1 (ICAM1);
   (cc) absence or presence of heat shock proteins;
   (dd) absence or presence of apoB, apoA, apoE;
   (ee) absence or presence of homocysteine or parts thereof;
   (ff) absence or presence of Streptococcus sp., Porphyromonas gingivalis, Helicobacter pylori and Chlamydia pneumoniae or immunological relatives thereof;
   (gg) absence or presence of necrosis and platelet markers;
   (hh) absence or presence of leptin;
   (ii) absence or presence of vasopeptidase inhibitor of cardiac endogenous kinins;
   (jj) absence or presence of heparin;
   (kk) absence or presence of metalloproteinase-9;
   (ll) absence or presence of metalloproteinase-1 including its tissue inhibitor;
   (mm) absence or presence of angiotensin-converting enzyme;
   (nn) absence or presence of CD95/Apo1/Fas;
   (oo) absence or presence of hepatocyte growth factor;
   (pp) absence or presence of soluble vascular cell adhesion molecule-1 (VCAM1);
   (qq) absence or presence of plasma brain natriuretic peptide;
   (rr) absence or presence of angiotensin II type receptor;
   (ss) absence or presence of endothelial constitutive nitric oxide synthase;
   (tt) absence or presence of glycoprotein IIIa genetic polymorphisms;
   (uu) absence or presence of factor VIIa;
   (vv) absence or presence of thrombin;
   (ww) absence or presence of endothelin-1;
   (xx) absence or presence of cardiac myofibrillar proteins;
   (yy) absence or presence of Fas and Fas ligand; and
   (zz) absence or presence of ligands thereof or binding partners thereof or nucleic acid molecules encoding same or their fragments or ligands or binding partners;
(2) adding the values for said features to provide a predictive value for said sequence; and
(3) outputting said predictive value.

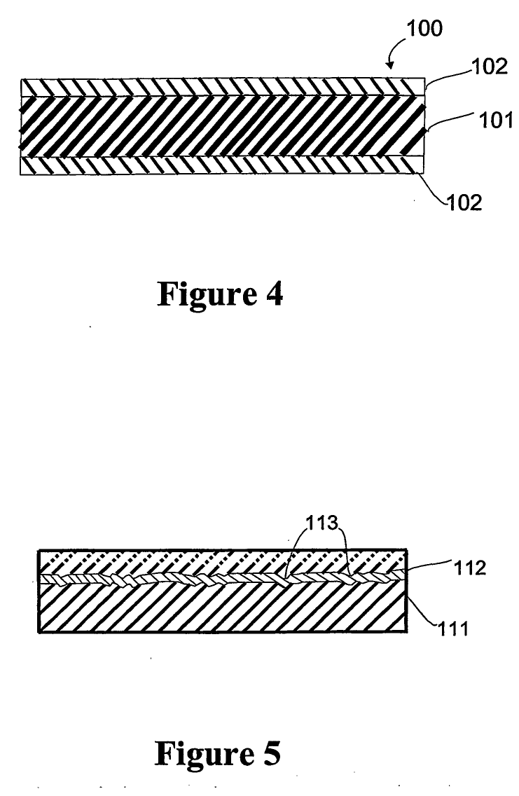

FIG. 4 shows a cross section of a magnetic data storage medium 100 which can be encoded with machine readable data, or a set of instructions, for designing a synthetic molecule of the invention, which can be carried out by a system such as system 10 of FIG. 5. Medium 100 can be a conventional floppy diskette or hard disk, having a suitable substrate 101, which may be conventional, and a suitable coating 102, which may be conventional, on one or both sides, containing magnetic domains (not visible) whose polarity or orientation can be altered magnetically. Medium 100 may also have an opening (not shown) for receiving the spindle of a disk drive or other data storage device 24. The magnetic domains of coating 102 of medium 100 are polarized or oriented so as to encode in manner which may be conventional, machine readable data such as that described herein, for execution by a system such as system 10 of FIG. 3.

FIG. 4 shows a cross section of an optically readable data storage medium 110 which also can be encoded with such machine-readable data, or set of instructions, for screening a candidate molecule of the present invention, which can be carried out by a system such as system 10 of FIG. 3. Medium 110 can be a conventional compact disk with read only memory (CD-ROM) or a rewritable medium such as a magneto-optical disk, which is optically readable and magneto-optically writable. Medium 100 preferably has a suitable substrate 111, which may be conventional, and a suitable coating 112, which may be conventional, usually of one side of substrate 111.

In the case of a CD-ROM, as is well known, coating 112 is reflective and is impressed with a plurality of pits 113 to encode the machine-readable data. The arrangement of pits is read by reflecting laser light off the surface of coating 112. A protective coating 114, which preferably is substantially transparent, is provided on top of coating 112.

In the case of a magneto-optical disk, as is well known, coating 112 has no pits 113, but has a plurality of magnetic domains whose polarity or orientation can be changed magnetically when heated above a certain temperature, as by a laser (not shown). The orientation of the domains can be read by measuring the polarisation of laser light reflected from coating 112. The arrangement of the domains encodes the data as described above.

The present system retrieves features and forms composite features from them. More than one feature can be combined in a variety of different ways to form these composite features. In particular, the composite feature can be any function or combination of a simple feature and other composite features. The function can be algebraic, logical, sinusoidal, logarithmic, linear, hyperbolic, statistical and the like. Alternatively, more than one feature can be obtained in a functional manner (e.g. arithmetic, algebraic). By way of example, a composite feature may equal the sum of two or more features or a composite feature may correspond to a sub-fraction of overlap of one or more features from another feature. Alternatively, a composite feature may equal a constant times one or more features. Of course, there are many other ways composite features can be defined.

The present invention is further described by the following non-limiting Examples.

### EXAMPLE 1

### Early Biochemical Markers of a Cardiac Condition or Event

Myoglobin, CK-MB, Cardiac Troponin-T and Cardiac Troponin-I

The prime requisite for an early marker is that the development of the test should be readily performed in a short time period and that the marker be released soon after the ACS including AMI. Ideally, an early diagnostic for ACS including AMI is a Point-of-Care device, namely a small, preferably hand-held device which is used when a potential ACS including AMI patient first presents at a clinic including a hospital or general physician's practice.

Of the early markers, each of myoglobin, total creatinee kinase (CK) including creatinee kinase MB isoform (CK-MB), cardiac troponin-T (cTn-T) and cardiac troponin-I (cTn-I), has slightly different characteristics based on:—
(1) whether it is specific to cardiac tissue;
(2) time to reach peak serum levels;
(3) the period during which peak level is maintained;
(4) clinical sensitivity, and
(5) clinical specificity (Dean, 1998).

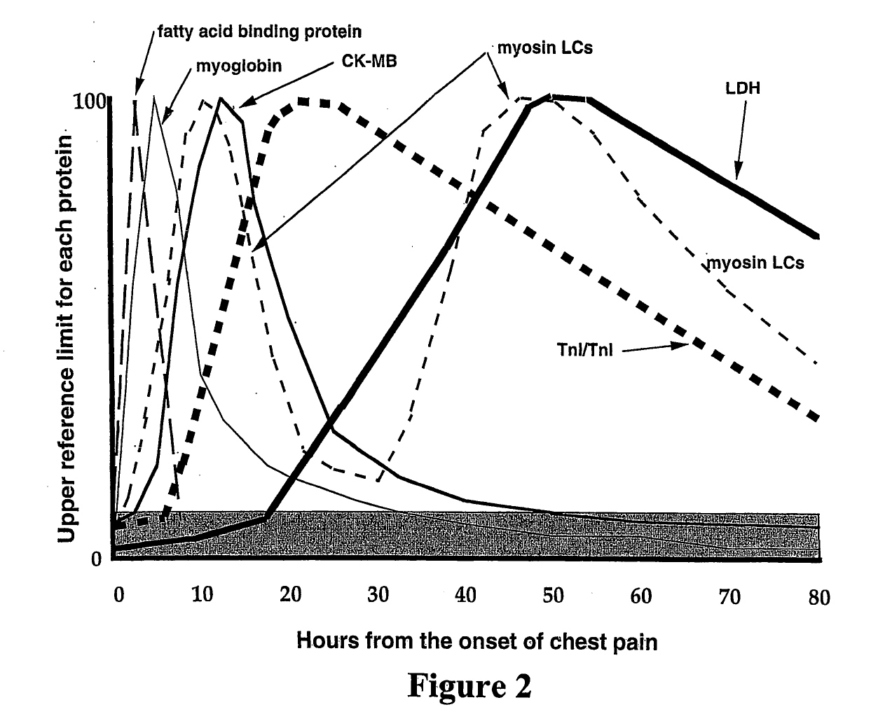

FIG. 2 shows multiple time courses of the appearance of cardiac markers.

Myoglobin is not cardiac-specific but is the first to reach a peak (FIG. 2). It is usually assayed by an immunoassay. Myoglobin assays are conveniently determined using either monoclonal or polyclonal antibodies or combinations of these. Serum levels peak at 4-5 hours from the onset of chest pain but the peak is transient and may fall to non-diagnostic levels after 10-12 hours. CK-MB is a reliable marker of ACS including AMI. It rises to peak levels at about 12 hours and falls rapidly to non-diagnostic levels at 20-24 hours after the onset of chest pain.

Both cTn-I and cTn-T are cardiac specific. cTN-I has an advantage since cTn-T may be expressed by skeletal muscle following experimental injury and has been detected in regenerating skeletal muscle in man (Bordet et al., 1997). cTn-T is, however, clinically quite sensitive and is capable of detecting 64% of true AMI patients. When the infarct is small, the cTn-T test can result in a significant number of false negative results. cTn-T has, however, an advantage of rising to significantly elevated levels at 5-11 hours from the onset of chest pain. It remains elevated and fairly constant for 5-8 days.

Chest pain is the commonest cause of presentations to Emergency Departments, but for 75% of patients with chest pain, the pain is non-cardiac in origin. Thus, it is also important to have a negative predictive value in tests for ACS to allow for safe early discharge or a safe decision not to admit a patient to hospital.

Most of the available early markers are interrelated. Thus, the difficulties faced by obtaining false-positives are largely overcome by correlating a test (e.g. CK-MB antibody test) with other tests such as the myoglobin test. At present, this is both inconvenient and expensive, especially where short turn-around times are required and specialist staff are needed to operate the diagnostic instruments in hospital biochemistry departments. They include CK-MB, cTn-T and cTn-I. The ability to test a number of early markers using the microarray method of the present invention is an advantage because the reliability is significantly increased.

Medium-Term and Long-Term Biochemical Markers

These markers peak at day 1.5-3 or later. They include total creatine kinase (CK), cardiac troponin I (cTn-I), aspartate aminotransferase (AST), lactate dehydrogenase (LDH), myosin light chain (MLC) and myosin heavy chain (MHC), fatty acid binding (FAB) protein and ABC. Like the early markers, the late markers can be used to estimate the size of the infarct. Multiple medium/long term markers are valuable for patients who present late and so cannot be tested for short-term markers.

Other serum markers are available such as fatty acid binding protein which appears to be a valuable early (<10 h) marker of AMI.

### EXAMPLE 2

### Evaluation of the Efficiency of the Diagnostic Performance of the Device

The efficiency (E) of the diagnostic assay is determined using the formula:

$$E = \frac{TP}{TO} \times 100$$

where TP=true positives and TO=total number of tests. TO is calculated by the expression TO=TP+FP+FN+TN where FP=false positives; FN=false negatives and TN=true negatives. E has the following range of values: 0<E≦100.

### EXAMPLE 3

### Estimation of Infarct Size

The magnitude of the change of a biochemical marker as a function of time correlates with the size of the infarct determined at autopsy. Clearly the reliability of these integrations is enhanced if a multiplicity of tests is available.

The size (volume) of an infarct is a function of:—
(1) the time scale over which the biochemical marker is released;
(2) the rate of release of the biochemical marker (f(t));
(3) the body weight (Bw) of the patient;
(4) the proportion of the body weight into which the biochemical marker is released (Kw);
(5) the rate of removal of the biochemical marker from circulation (Ed);
(6) the total biochemical marker released divided by the amount of that marker released from the infarcted myocardium (Kr).

Accordingly, infarct size (Is) is determined using the formula:—

$$Is = \frac{{\int }_{0}^{t} f ( t ) ⅆ t \times Bw \times Kw}{Ed \times Kr}$$

### EXAMPLE 4

### Assessment of Agents which Contribute to or are Associated with Myocardial Inflammation

The incidence of cardiovascular diseases has been statistically connected to certain infectious agents. Certain microorganisms including bacteria (for example, Porphyromonas and Chlamydia), certain bacteria (for example, Helicobacter pylori, is probably the most widespread disease-causing agent), and certain viruses (Coxsackie virus and Cytomegalo virus) have a contributing effect on the propensity for cardiovascular disease including ACS such as AMI. These agents may achieve their effects on the cardiovascular system through an inflammatory monocyte.

The presence of these microorganisms and viruses is detected using antibodies directed against specific epitopes on the surfaces of these pathogens.

### EXAMPLE 5

### The Nature of the Biochemical Marker Device

The present invention provides a device which simultaneously determines a large number of antigens from a sample of body fluid. The combination of biochemical markers for ACS including AMI provides an unequivocal diagnosis.

The test is performed on a small (10-100 microlitres) volume of serum enabling the measurement to be performed several times within a short period to minimize errors providing reliable diagnosis of ACS.

The invention may also assay other body fluids (such as saliva) where pathogens (such as Porphyromonas and other microorganisms) reside and thus provide evidence for the origin of potential inflammatory responses.

As the patterns of genetic susceptibility of ACS and congestive cardiac failure (CCF) including dilated cardiomyopathy (DCM) are known, the test is modifiable to include mRNA and cDNA markers for DNA derived from patient leukocytes to evaluate their genetic propensity for cardiovascular disease.

### EXAMPLE 6

### Adsorption or Coupling of Antibodies as an Array on a Solid Support

Specific antibodies for marker proteins (for example, proteins derived from heart tissue) are trapped or immobilized on a solid surface. Immobilization of protein antibodies also may be achieved by electrostatic forces or by the assistance of streptavidin attached to activated spots on the solid support to which biotinylated antibodies can attach. The volume of antibody used to form the capturing spot is small, approximately 10 pL-100 nanolitres. They are allowed to dry in the presence of protecting chemicals such as arabinose and low molecular weight (3000-5000 Da) polyethylene glycol. A micro-array of antibodies specific for cardiac markers is constructed in two dimensions by application of tiny (10 pL-10 nL) spots of each antibody at addressable locations on a solid support using a high precision applicator such as a Biodot 3000 (Cartesian Technologies, Irvine, Calif., USA). Antibodies are adsorbed to a surface of nylon or nitro-cellulose, or covalently linked to a membrane such as Immobilon P (Millipore Corporation) using established procedures. The solid supports are generally immersed or otherwise contacted with blocking agents such as but not limited to skim milk powder, Irish moss extracts, or other sources of carrageenan or gelatin.

There are several options for detecting antigens in plasma from a blood sample which have bound to an antibody array (FIG. 1):—
(i) The plasma is reacted with N-hydroxy succinimide biotin or N-ethyl maleimide biotin which covalently modify lysine and cysteine residues, respectively.

Components of low molecular weight in the plasma, including residual modification reagents, are removed by centrifugal desalting through a molecular sieve such as Sephadex G-10. The biotinylated proteins in the desalted plasma are then applied to an array of antibodies against cardiac markers and incubated for 30 min at 37° C. Unbound protein solution is decanted and the array is washed several times with phosphate buffered saline (PBS). Biotinylated proteins bound to antibody spots are detected by application of a conjugate of streptavidin with horse radish peroxidase (HRP), streptavidin with alkaline phosphatase (AP) [FIG. 1b], or streptavidin modified with fluoroscein (FITC) or Texas Red. The most sensitive forms of detection with HRP and AP involve the enzymic synthesis of products which are chemiluminescent. Substrate kits which produce chemiluminescence from bound HRP or AP activity are available from Amersham Pharmacia Biotech (Little Chalfont, UK), Bio-Rad Laboratories (Hercules, USA) and Pierce Chemical Company (Rockford, USA). After application of the substrate to the array, the moist membrane is put in contact with X-ray film for 2-5 min and then developed to reveal the antibody spots which bound a cardiac marker. Biotinylated cardiac markers bound to the array could be visualized directly by application of streptavidin modified with fluoroscein (FITC) or Texas Red. The intensity of the spots on the array using one of these three procedures could be quantified using a densitometric scanner (e.g. from Molecular Dynamics, Sunnyvale, Calif., USA) or fluorometric scanner (e.g. STORM, Molecular Dynamics), enabling calculation of concentrations of these proteins in the blood sample.
(ii) Alternatively, the plasma is reacted with a reagent which covalently attaches fluorescent groups to amino or sulfhydryl groups on all proteins in the sample (FIG. 1e). Suitable fluorophores available as protein labelling kits are Alexa 488 and CBQCA which may be excited at 488 nm using an argon laser. Fluorescently-labelled plasma proteins bound to an antibody array could be quantified using a scanning fluorimeter or a confocal microscope. Mild reaction conditions should be used so that the majority of antigen binding sites are not affected. Different cardiac markers will be labelled to different extents with different numbers of fluorophores. Plasma samples may need to be dialyzed prior to reaction with the fluorophore, and should be desalted after chemical derivatization.
(iii) Unlabelled antigens bound to an array are reactable with soluble, fluorescently-labelled antibodies which bind to a different epitope of the bound cardiac marker, enabling quantitation of bound antigens (FIG. 1e). For many samples, the level of plasma antigen may exceed the number of antibodies available in a particular spot. Positive antigens of interest from an initial screen may be quantitated subsequently using a row of dilutions of a particular antibody. When the number of antibodies in a spot on this array exceed the number of antigens applied in the sample, there would be no further increase in fluorescence. A plot of fluorescence versus antibody level would give the level of antigen in the plasma sample. Levels of bound antigens on this quantitative array could be determined using procedures (i) or (ii) above or the number of vacant antibodies on the array could be determined using standard, fluorescently-labelled antigen.

### EXAMPLE 7

### Detecting the Onset of AMI

The device provides an indication of the time of onset of the AMI even independent of whether the AMI is sensed by the patient (onset of chest pain) or whether it is a silent infarct associated with a test such as a stress test. Patients are sampled at comparatively high frequencies resulting in an improved definition of the shape of the biochemical marker curve (FIG. 2). The results of the test may be skewed to place greater weight on antibodies that proved to be the most sensitive in any particular patient. Combinations of tests for early markers provides a better basis for determining the onset of AMI. If a patient presents later (>24 hours following the onset of chest pain), the time of onset is determined using late markers in the array.

### EXAMPLE 9

### Detecting the Size of the Infarct

Cardiac myosin heavy chain (cMHC) is released after a prolonged delay and does not appear in the serum for about two days after an AMI. It is considered to be a reliable marker for estimating infarct size (Mair et al, 1994). When cardiac troponin-T (cTn-T) has a late peak, the serum concentrations correlate well with estimates based on radionucleoide imaging methods for infarct size. Cardiac myosin light chain (MLC) levels also correlate with infarct size (Omura et al., 1995).

### EXAMPLE 10

### Detecting Myocardial Reperfusion after an Acute Coronary Syndrome Incident

When a patient does present with acute coronary syndrome such as AMI, very often the therapeutic aim is to re-open an occluded coronary artery. This is usually done by thrombolytic agents such as TPA or by mechanical means, such as balloon angioplasty, with or without stenting. The aim of these therapies is to achieve reperfusion of the threatened myocardium. It is often difficult, however, to determine whether reperfusion has been successful, both in terms of the time of reperfusion and the extent of myocardium successfully salvaged. At the moment, the clinical tools for assessing reperfusion are insensitive and non-specific. They include clinical features, such as the disappearance of chest pain, and ECG features such as decrease in the amount of ST segment elevation. Serum markers are sometimes helpful, but usually only in retrospect.

Therefore, reperfusion is not easily diagnosed in current clinical practice, but is of great interest. After successful reperfusion, there is often a “washout” phase, where the level of traditional markers of cardiac injury such as CK-MB and troponin rise very dramatically for a short period of time. It may be that the pattern of release of markers, and/or the ratio of one marker to another during this release phase, may give informative data about the timing and extent of reperfusion. This is another possible application of the proposed methodology.

### EXAMPLE 11

### Detecting the Presence of Pathogens Associated with Myocardial Inflammation

Infection may be a cofactor in the formation of atherosclerosis. Infectious agents such as microorganisms (Chlamydia, Porphyromonas, Helicobacter and Streptococcus) and certain viruses (Cytomegalovirus, Coxsackie virus) may be detected using existing immunological methods which can be adapted for use in the cardiac microarray device.

### EXAMPLE 12

### Determination of Coronary Risk Factors

The risk of atherosclerotic disease can be evaluated using a variety of serum markers. Of these, the following is a list of potential apolipoproteins, lipoproteins, enzymes, receptors and transfer proteins that can be monitored using antibody based microarrays.

Plasma lipoproteins are characterized according to their density, flotation rate, mean diameter and electrophoretic mobility. The major classes of plasma lipoprotein include: chylomicrons, VLDL (pre β-lipoprotein), LDL (β-lipoprotein) and HDL. The apoprotein compositions which characterize these classes of lipoproteins are set out in Table 1.

TABLE 1

| | | Chylomicron | VLDL | LDL | HDL |
|---|---|---|---|---|---|
| Apoproteins | MW (Da) | (% of total protein) | | | |
| ApoA-I | 28,300 | trace | trace | trace | 66 |
| ApoA-II | 17,000 | trace | trace | trace | 20 |
| ApoB₁₀₀ | 512,000 | 5-20 | 37 | 97 | — |
| ApoC-I | 6,631 | 15 | 7 | trace | 3 |
| ApoC-II | 8,851 | 15 | 7 | trace | trace |
| ApoC-III | 8,864 | 40-50 | 40 | 2 | 4 |
| ApoD | — | — | — | — | 5 |
| ApoE | ˜34,000 | 4 | 13 | 1 | 1 |

Apolipoproteins help to solubilize cholesterol esters and triglycerides by interacting with phospholipids. Apoproteins bind lipids largely on hydrophobic interaction between the fatty acyl chains of phospholipids and the non-polar regions of the apoproteins and to a lesser extend on the ionic interactions of the charged head groups of phospholipids and oppositely charged regions of the apoprotein.

Apolipoprotein A (ApoA-I and ApoA-II) is the major component of HDL. It can be subdivided into ApoA-I (MW 28,300 Da) and ApoA-II (MW 17,000 Da).

Apolipoprotein B (ApoB) is heterogeneous. ApoB₁₀₀ is mainly found in chylomicrons, VLDL and LDL. ApoB₄₈ represents the amino terminal half of ApoB₁₀₀ and is not a ligand for the LDL receptor.

Apolipoproetin C (ApoC) comprises at least three components, which occur as major components of VLDL and as minor components of HDL. ApoC-1 has a MW of 6,631 Da, ApoC-II has a MW of 8,837 Da and ApoC-III is 8,767 Da. ApoC-III also has three isoforms depending on whether the protein has 0, 1 or 2 sialic acid residues in its carbohydrate moiety.

Apolipoprotein D (ApoD) is a minor component of HDL and is not normally considered to be a useful plasma marker.

Apolipoprotein E (ApoE) is found in VLDL, LDL (particles formed during the conversion of VLDL to LDL) and HDL. This protein is heterogeneous and has a MW of approximately 34,000 Da. There are several subtypes including ApoE2.

Thus, the following antibodies to apolipoproteins can be used to monitor chylomicrons, VLDL, LDL and HDL (see Alaupovic et al., 1971):—
ApoA-I (for HDL) available from Research Diagnostics Inc. (RDI-PRO6182);
ApoB₁₀₀ (for LDL) available from Research Diagnostics Inc (RI-PRO610800 and RDI-OXLDLabm for the oxidized form of LDL);
ApoC-I (for chylomicrons and to a lesser extent VLDL and HDL);
ApoC-II (for chylomicrons and to a lesser extent VLDL);
ApoC-III (for chylomicrons and VLDL and to a lesser extent LDL and HDL);
ApoD available from Research Diagnostics Inc. (RDI-APODabm) and
ApoE (for VLDL and to a lesser extent chylomicrons) available from Research Diagnostics Inc. (RDI-PRO61085/61086 and RDI-APOEabGX) and ApoE2 to identify ApoE2 (RDI-APO61088).

Apolipoproteins are detected with a “sandwich” assay. Two types of assay can be conducted:—
(i) An antibody-based capture assay: This is a sandwich assay where a specific polyclonal or monoclonal antibody is used to capture an apolipoprotein antigen which is then detected using a second antibody conjugated to a detector/reporter moiety such as horse radish peroxidase (HRP) which is then visualized by developing a colour reaction; and
(ii) A receptor-based capture assay; Here, the apolipoprotein is captured by a specific receptor protein and then specifically identified using a secondary antibody conjugated to a reporter molecule such as BRP and detected as above.

The antibody-based capture assay can be performed using an immobilized antibody on a nitrocellulose-coated glass microscope slide or other solid surface as follows:—
(1) purified antibodies are diluted in PBS (0.1 M sodium phosphate buffer at pH 7.2, 0.137 M sodium chloride) or used undiluted and applied to the nitrocellulose to form a rectangular array using the BioDot arrayer and left overnight at 4° C.;
(2) the excess antibody is removed by three washes with PBS and the dots are allowed to dry,
(3) a small volume of plasma (˜0.1 ml diluted up to 1:100 or a standard) appropriately diluted is then applied to the array and incubated at 37° C. for 1-2 hours;
(4) the antibody array is then washed with PBS and allowed to dry;
(5) add a second polyclonal antibody (preferably directed against the apolipoprotein conjugated to biotin peroxidase diluted up to 1:15,000 in PBS containing 1% bovine serum albumin (BSA);
(6) incubate at 37° C. for two hours;
(7) wash;
(8) incubate the array with substrate (3 g o-phenylenediamine dihydroxychloride in phosphate buffered citrate pH 5.5 containing 3.5 mM hydrogen peroxide) for 30 min (in the dark);
(9) stop the reaction with 0.1 M HCl; and
(10) read the OD_{492 nm} and compare the plasma samples with a sample in which the apolipoprotein was omitted. Usually, ApoB is used as a standard lipoprotein where the ApoB is precipitated using isopropanol.

The receptor-based capture assay is performed as above except that the antibody in step (1) is replaced with a recombinant receptor protein and the second antibody used in step (5) is replaced by a primary antibody directed against the particular apolipoprotein.

As well as the apolipoproteins associated with the chylomicrons, HDLs, VLDL and LDL, there is a series of associated enzymes in the plasma (for example, lipoprotein lipase (LPL), hepatic lipase (HPL) and lecithin cholesterol acyltransferase (LCAT, 60 kDa) which may be detected using specific antibodies.

### EXAMPLE 13

### Other Circulating “Risk-Factor” Proteins

The following proteins may be detected in significantly altered amounts in the serum of patients who are considered to be “at risk” of a cardiac aberration (see al Audelmottalab et al., 1999):—
(i) glycosylated haemoglobin (Hba1c);
(ii) insulin;
(iii) fibrinogen;
(iv) Factor VII;
(v) soluble ICAM-1; and
(vi) C-reactive protein (C protein).

These proteins can be monitored using the antibody-based capture assay described above.

### EXAMPLE 14

### Preparation of the Array

The array may include all known biochemical markers of acute myocardial infarction as well as factors known to be associated with coronary disease. The array may comprise monoclonal antibodies covalently linked to a membrane as multiple spots in a 2-dimensional matrix.

The spots are of a microscopic size, resulting from the application of a very small drop of antibody solution (e.g. 10 nanolitres) and are well separated from adjacent spots (100 microns separating the dots).

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Abdelmottaleb et al., Am. Heart J. 137: 346-51, 1999
Alupovic, Atherosclerosis 13: 141, 1971
Border et al., Clin. Chem. 43: 476-484, 1997
Dean, Cardiac Troponin-Tas a marker of myocardial injury. In Cardiac Markers (ed AHB Wu) Humana Press, Totowa N.J.) pp 205-227, 1998
Heid et al., 1996, Genome Res. 6: 986-994
Herzberg and Meyer, M. W., Ann. Periodontal. 3: 151-60, 1998
Mayr et al. Circulation 99: 1560-6, 1999
Omura et al., Int. Jpn. Circ. J. 59: 154-9, 1995

1. A method of stratifying a subject on the basis of:
(i) having experienced a prior event of the vasculature (acute);
(ii) having not experienced a prior event of the vasculature; and/or
(iii) having experienced a prior event of the vasculature resulting in a level of disability selected from Class I to Class IV (chronic);
wherein:
Class I comprises subjects with coronary artery disease (CAD) and other disorders where ordinary physical activity does not cause fatigue, palpitations, shortness of breath or anginal (heart) pain;
Class II comprises subjects as in Class I but where subjects are comfortable at rest but where there is slight limitation resulting from exercise;
Class III comprises subjects as in Class II but where there is now a marked limitation to physical activity; and
Class IV comprises subjects as in Class III but where any physical activity causes angina and discomfort;
said method comprising contacting a sample from said subject which comprises members which are present, absent, elevated or otherwise activated or up- or down-regulated in the subject prior to, during or following said event of the vasculature with one or more binding partners of said members wherein the binding partners are immobilized to a solid support and wherein the pattern of interaction between the members and their binding partners provides an assessment of the presence or absence or actual level or change in level of the event of the vasculature such that:—
(i) the presence or actual level or elevated levels of at least five members selected from myoglobin, myosin light chain, myosin heavy chain, total creatine kinase, total creatine kinase-MB, lactate dehydrogenase, aspartate aminotransferase, cardiac troponin I, cardiac troponin T, fatty acid binding protein (FABP), glycogen phosphorylase-BB isoenzyme, α-atrial natriuretic peptide, brain natriuretic peptide, adrenomedullin, low density lipoprotein, very low density lipoprotein, high density lipoprotein and intermediate density lipoprotein, C reactive protein, serum amyloid A, P-selectin, prostaglandins, platelet-activating factor, histamine, tumor necrosis factor α, soluble TNF receptor 2, fibrin, fibrinogen, fibrinolytic peptides, modified haemoglobin, ferritin, soluble intercellular adhesion molecule including soluble intercellular adhesion molecule-1, heat shock proteins, apoB, apoA, apoE, homocysteine or parts thereof, Streptococcus sp., Porphyromonas gingivalis, Helicobacter pylori and Chlamydia pneumoniae or immunological relatives thereof, necrosis and platelet markers, leptin, vasopeptidase inhibitor of cardiac endogenous kinins, heparin, metalloproteinase-9, metalloproteinase-1 including its tissue inhibitor, angiotensin-converting enzyme, CD95/Apo1/Fas, hepatocyte growth factor, soluble vascular cell adhesion molecule-1, plasma brain natriuretic peptide, angiotensin II type receptor, endothelial constitutive nitric oxide synthase, glycoprotein IIIa genetic polymorphisms, factor VIIa, thrombin, endothelin-1, cardiac myofibrillar proteins, Fas and Fas ligand, ligands thereof or binding partners thereof or nucleic acid molecules encoding same or their fragments or ligands or binding partners;
wherein at least one of the five members is selected from myoglobin, total creatine kinase, total creatine kinase-MB, lactate dehydrogenase, cardiac troponin-I, cardiac troponin-T, brain natriuretic peptide and C reactive protein;
is indicative of an acute condition;
(ii) the absence of or no change in the level of a combination of the members listed in (i) is indicative of the absence of an event of the vasculature; and
(iii) the presence or actual level or an elevation of at least 10 members selected from myoglobin, myosin light chain, myosin heavy chain, total creatine kinase, total creatine kinase-MB, lactate dehydrogenase, aspartate aminotransferase, cardiac troponin I, cardiac troponin T, fatty acid binding protein, (FABP), glycogen phosphorylase-BB isoenzyme, α-atrial natriuretic peptide, brain natriuretic peptide, adrenomedullin, low density lipoprotein, very low density lipoprotein, high density lipoprotein and intermediate density lipoproteinC reactive protein, serum amyloid A, P-selectin, prostaglandins, platelet-activating factor, histamine, tumor necrosis factor α, soluble TNF receptor 2, fibrin, fibrinogen, fibrinolytic peptides, modified haemoglobin, ferritin, soluble intercellular adhesion molecule including soluble intercellular adhesion molecule-1, heat shock proteins, apoB, apoA, apoE, homocysteine or parts thereof, Streptococcus sp., Porphyromonas gingivalis, Helicobacter pylori and Chlamydia pneumoniae or immunological relatives thereof, necrosis and platelet markers, leptin, vasopeptidase inhibitor of cardiac endogenous kinins, heparin, metalloproteinase-9, metalloproteinase-1 including its tissue inhibitor, angiotensin-converting enzyme, CD95/Apo1/Fas, hepatocyte growth factor, soluble vascular cell adhesion molecule-1, plasma brain natriuretic peptide, angiotensin II type receptor, endothelial constitutive nitric oxide synthase, glycoprotein IIIa genetic polymorphisms, factor VIIa, thrombin, endothelin-1, cardiac myofibrillar proteins, Fas and Fas ligand, ligands thereof or binding partners thereof or nucleic acid molecules encoding same or their fragments or ligands or binding partners, is indicative of a chronic condition.

2. The method of claim 1 wherein the event associated with the vasculature is selected from vascular disease, cardiovascular disease, stroke, pulmonary, renal vascular, cerebrovascular, thrombotic or generalized arterial or venous condition or event, organ failure including liver, kidney or heart failure, tissue rejection such as organ transplant rejection, a thrombotic event including deep vein thrombosis, an infection, damage to vessels of the circulatory system, stent failure or trauma caused by a stent, pace-maker or other prosthetic device, vascularization of a tumor, events or conditions following surgery, trauma or age-related disease and endothelial damage.

3. The method claim 1 wherein the event associated with the vasculature is a cardiovascular condition.

4. The method of claim 3 wherein the cardiovascular condition is acute coronary syndrome.

5. The method of claim 1 wherein the binding partners of the members of the biological sample are immunointeractive molecules.

6. The method of claim 5 wherein the immunointeractive molecules are antibodies.

7. The method of claim 6 wherein binding of members to binding partners is detected by a labeled antibody specific to the member in the biological sample.

8. The method according to claim 6 wherein the binding of a binding member to a binding partner is detected by a method selected from:—
(i) biotinylation of all plasma proteins, which are then bound to an antibody microarray and then to a streptavidin-AP/HRP conjugate;
(ii) fluorescent labeling of all plasma proteins;
(iii) fluorescently-labeled antibodies specific for a different epitope; and
(iv) concentrations of plasma markers determined using dilutions of immobilized antibodies.

9. The method of claim 1 for co-determining stratification based on whether or not a subject has experienced a recent or prior vascular related tissue injury, vasculature insufficiency or infarct or a related condition wherein the size of the vascular related tissue injury, vasculature insufficiency or infarct is determined by the formula:—
$$Is = \frac{{\int }_{0}^{t} f ( t ) ⅆ t \times Bw \times Kw}{Ed \times Kr}$$
wherein
Is is a vascular related tissue injury, vasculature insufficiency or infarct size;
f(t)dt is the rate of release of a member in a biological sample, said member being present, absent, elevated or otherwise activated in a subject following a cardiovascular condition or event leading to the vascular related tissue injury, vasculature insufficiency or infarct;
Bw is the body weight of the subject;
Kw is the proportion of the body weight into which the member is released;
Ed is the rate of removal of the member from evaluation; and
Kr is the total amount of member released divided by the amount of the member released from the vascular or infarcted tissue;
said method comprising contacting a biological sample from said subject wherein said sample comprises members present, absent, elevated or otherwise activated in a subject following a cardiovascular condition or event or a condition or event otherwise associated with a cardiovascular aberration with one or more binding partners of said members wherein the binding partners are immobilized to a solid support and wherein the pattern of interaction between the members and binding partners is indicative of whether the subject is acute or chronic with respect to the infarct wherein members of the biological sample are selected from five or more of myoglobin, myosin light chain, myosin heavy chain, total creatine kinase, total creatine kinase-MB, lactate dehydrogenase, aspartate aminotransferase, cardiac troponin I, cardiac troponin T, fatty acid binding protein (FABP), glycogen phosphorylase-BB isoenzyme, α-atrial natriuretic peptide, brain natriuretic peptide, adrenomedullin, low density lipoprotein, very low density lipoprotein, high density lipoprotein and intermediate density lipoprotein, C reactive protein, serum amyloid A, P-selectin, prostaglandins, platelet-activating factor, histamine, tumor necrosis factor α, soluble TNF receptor 2, fibrin, fibrinogen, fibrinolytic peptides, modified haemoglobin, ferritin, soluble intercellular adhesion molecule including soluble intercellular adhesion molecule-1, heat shock proteins, apoB, apoA, apoE, homocysteine or parts thereof, Streptococcus sp., Porphyromonas gingivalis, Helicobacter pylori and Chlamydia pneumoniae or immunological relatives thereof, necrosis and platelet markers, leptin, vasopeptidase inhibitor of cardiac endogenous kinins, heparin, metalloproteinase-9, metalloproteinase-1 including its tissue inhibitor, angiotensin-converting enzyme, CD95/Apo1/Fas, hepatocyte growth factor, soluble vascular cell adhesion molecule-1, plasma brain natriuretic peptide, angiotensin II type receptor, endothelial constitutive nitric oxide synthase, glycoprotein IIIa genetic polymorphisms, factor VIIa, thrombin, endothelin-1, cardiac myofibrillar proteins, Fas and Fas ligand, ligands thereof or binding partners thereof or nucleic acid molecules encoding same or their fragments or ligands or binding partners;
wherein:
(i) the presence or actual level or elevated levels of at least five members selected from myoglobin, myosin light chain, myosin heavy chain, total creatine kinase, total creatine kinase-MB, lactate dehydrogenase, aspartate aminotransferase, cardiac troponin I, cardiac troponin T, fatty acid binding protein (FABP), glycogen phosphorylase-BB isoenzyme, α-atrial natriuretic peptide, brain natriuretic peptide, adrenomedullin, low density lipoprotein, very low density lipoprotein, high density lipoprotein and intermediate density lipoprotein, C reactive protein, serum amyloid A, P-selectin, prostaglandins, platelet-activating factor, histamine, tumor necrosis factor α, soluble TNF receptor 2, fibrin, fibrinogen, fibrinolytic peptides, modified haemoglobin, ferritin, soluble intercellular adhesion molecule including soluble intercellular adhesion molecule-1, heat shock proteins, apoB, apoA, apoE, homocysteine or parts thereof, Streptococcus sp., Porphyromonas gingivalis, Helicobacter pylori and Chlamydia pneumoniae or immunological relatives thereof, necrosis and platelet markers, leptin, vasopeptidase inhibitor of cardiac endogenous kinins, heparin, metalloproteinase-9, metalloproteinase-1 including its tissue inhibitor, angiotensin-converting enzyme, CD95/Apo1/Fas, hepatocyte growth factor, soluble vascular cell adhesion molecule-1, plasma brain natriuretic peptide, angiotensin II type receptor, endothelial constitutive nitric oxide synthase, glycoprotein IIIa genetic polymorphisms, factor VIIa, thrombin, endothelin-1, cardiac myofibrillar proteins, Fas and Fas ligand, ligands thereof or binding partners thereof or nucleic acid molecules encoding same or their fragments or ligands or binding partners;
wherein at least one of the five members is selected from myoglobin, total creatine kinase, total creatine kinase-MB, lactate dehydrogenase, cardiac troponin-I, cardiac troponin-T, brain natriuretic peptide and C reactive protein;
is indicative of an acute condition;
(ii) the absence of or no change in the level of a combination of the members listed in (i) is indicative of the absence of an event of the vasculature; and
(iii) the presence or actual level or an elevation of at least 10 members selected from myoglobin, myosin light chain, myosin heavy chain, total creatine kinase, total creatine kinase-MB, lactate dehydrogenase, aspartate aminotransferase, cardiac troponin I, cardiac troponin T, fatty acid binding protein (FABP), glycogen phosphorylase-BB isoenzyme, α-atrial natriuretic peptide, brain natriuretic peptide, adrenomedullin, low density lipoprotein, very low density lipoprotein, high density lipoprotein and intermediate density lipoprotein, C reactive protein, serum amyloid A, P-selectin, prostaglandins, platelet-activating factor, histamine, tumor necrosis factor α, soluble TNF receptor 2, fibrin, fibrinogen, fibrinolytic peptides, modified haemoglobin, ferritin, soluble intercellular adhesion molecule including soluble intercellular adhesion molecule-1, heat shock proteins, apoB, apoA, apoE, homocysteine or parts thereof, Streptococcus sp., Porphyromonas gingivalis, Helicobacter pylori and Chlamydia pneumoniae or immunological relatives thereof, necrosis and platelet markers, leptin, vasopeptidase inhibitor of cardiac endogenous kinins, heparin, metalloproteinase-9, metalloproteinase-1 including its tissue inhibitor, angiotensin-converting enzyme, CD95/Apo1/Fas, hepatocyte growth factor, soluble vascular cell adhesion molecule-1, plasma brain natriuretic peptide, angiotensin II type receptor, endothelial constitutive nitric oxide synthase, glycoprotein IIIa genetic polymorphisms, factor VIIa, thrombin, endothelin-1, cardiac myofibrillar proteins, Fas and Fas ligand, ligands thereof or binding partners thereof or nucleic acid molecules encoding same or their fragments or ligands or binding partners is indicative of a chronic condition.

10. The method of claim 9 wherein the binding partners of the members of the biological sample are immunointeractive molecules.

11. The method of claim 10 wherein the immunointeractive molecules are antibodies.

12. The method of claim 9 wherein binding of members to binding partners is detected by a labeled antibody to the member in the biological sample.

13. The method of claim 12 wherein the binding of a binding member to a binding partner is detected by a method selected from:—
(i) biotinylation of all plasma proteins, which are then bound to an antibody microarray and then to a streptavidin-AP/HRP conjugate;
(ii) fluorescent labeling of all plasma proteins;
(iii) fluorescently-labeled antibodies specific for a different epitope; and
(iv) concentrations of plasma markers determined using dilutions of immobilized antibodies.

14. An array of binding partners for members in a biological sample from a subject said members being present, absent, elevated or otherwise activated in a subject following vasculature related tissue injury, vasculature insufficiency or infarct of a size determined by the formula:—
$$Is = \frac{{\int }_{0}^{t} f ( t ) ⅆ t \times Bw \times Kw}{Ed \times Kr}$$
wherein
Is is a vasculature related tissue injury, vasculature insufficiency or infarct size;
f(t)dt is the rate of release of a member in a biological sample, said member being present, absent, elevated or otherwise activated in a subject following a cardiovascular condition or event leading to the vasculature related tissue injury, vasculature insufficiency or infarct;
Bw is the body weight of the subject;
Kw is the proportion of the body weight into which the member is released;
Ed is the rate of removal of the member from evaluation; and
Kr is the total amount of member released divided by the amount of the member released from the vasculature related tissue injury, vasculature insufficiency or infarcted tissue;
wherein the binding partners are defined by (x,y) coordinates such that the array comprises n binding partners at coordinates (x,y), (x₂, y₂) . . . (xₙ,yₙ) and wherein the pattern of interaction between the members and the binding partners is indicative of said infarct or otherwise provides data input for assessment of the size of the infarct wherein members of the biological sample are selected from five or more of myoglobin, myosin light chain, myosin heavy chain, total creatine kinase, total creatine kinase-MB, lactate dehydrogenase, aspartate aminotransferase, cardiac troponin I, cardiac troponin T, fatty acid binding protein (FABP), glycogen phosphorylase-BB isoenzyme, α-atrial natriuretic peptide, brain natriuretic peptide, adrenomedullin, low density lipoprotein, very low density lipoprotein, high density lipoprotein and intermediate density lipoprotein, C reactive protein, serum amyloid A, P-selectin, prostaglandins, platelet-activating factor, histamine, tumor necrosis factor α, soluble TNF receptor 2, fibrin, fibrinogen, fibrinolytic peptides, modified haemoglobin, ferritin, soluble intercellular adhesion molecule including soluble intercellular adhesion molecule-1, heat shock proteins, apoB, apoA, apoE, homocysteine or parts thereof, Streptococcus sp., Porphyromonas gingivalis, Helicobacter pylori and Chlamydia pneumoniae or immunological relatives thereof, necrosis and platelet markers, leptin, vasopeptidase inhibitor of cardiac endogenous kinins, heparin, metalloproteinase-9, metalloproteinase-1 including its tissue inhibitor, angiotensin-converting enzyme, CD95/Apo1/Fas, hepatocyte growth factor, soluble vascular cell adhesion molecule-1, plasma brain natriuretic peptide, angiotensin II type receptor, endothelial constitutive nitric oxide synthase, glycoprotein IIIa genetic polymorphisms, factor VIIa, thrombin, endothelin-1, cardiac myofibrillar proteins, Fas and Fas ligand, ligands thereof or binding partners thereof or nucleic acid molecules encoding same or their fragments or ligands or binding partners.

15. The array of claim 14 wherein the binding partners of the members in the biological sample are immunointeractive molecules.

16. The array of claim 15 wherein the immunointeractive molecules are antibodies.

17. The array of claim 13 wherein the second set of members are immobilized to a solid support.

18. A computer program product for assessing a subject on the basis of:
(i) having experienced a prior event of the vasculature (acute);
(ii) having not experienced a prior event of the vasculature; and/or
(iii) having experienced a prior event of the vasculature resulting in a level of disability selected from Class I to Class IV (chronic);
wherein:
Class I comprises subjects with coronary artery disease (CAD) and other disorders where ordinary physical activity does not cause fatigue, palpitations, shortness of breath or anginal (heart) pain;
Class II comprises subjects as in Class I but where subjects are comfortable at rest but where there is slight limitation resulting from exercise;
Class III comprises subjects as in Class II but where there is now a marked limitation to physical activity; and
Class IV comprises subjects as in Class III but where any physical activity causes angina and discomfort;
said product comprising:—
(1) code that receives an input value for one or more of features wherein said features are selected from:—
(a) absence or presence or actual level or change in level of myoglobin;
(b) absence or presence or actual level or change in level of myosin light chain;
(c) absence or presence or actual level or change in level of myosin heavy chain;
(d) absence or presence or actual level or change in level of total creatine kinase;
(e) absence or presence or actual level or change in level of total creatine kinase-MB;
(f) absence or presence or actual level or change in level of lactate dehydrogenase;
(g) absence or presence or actual level or change in level of aspartate aminotransferase;
(h) absence or presence or actual level or change in level of cardiac troponin I and cardiac troponin T;
(i) absence or presence or actual level or change in level of fatty acid binding protein (FABP);
(j) absence or presence or actual level or change in level of glycogen phosphorylase-BB isoenzyme;
(k) absence or presence or actual level or change in level of α-atrial natriuretic peptide;
(l) absence or presence or actual level or change in level of brain natriuretic peptide;
(m) absence or presence or actual level or change in level of adrenomedullin;
(n) absence or presence or actual level or change in level of low density lipoprotein, very low density lipoprotein, high density lipoprotein and intermediate density lipoprotein;
(o) absence or presence or actual level or change in level of C reactive protein;
(p) absence or presence or actual level or change in level of serum amyloid A;
(q) absence or presence or actual level or change in level of P-selectin;
(r) absence or presence or actual level or change in level of prostaglandins;
(s) absence or presence or actual level or change in level of platelet-activating factor;
(t) absence or presence or actual level or change in level of histamine;
(u) absence or presence or actual level or change in level of tumor necrosis factor α;
(v) absence or presence or actual level or change in level of soluble TNF receptor 2;
(w) absence or presence or actual level or change in level of fibrin;
(x) absence or presence or actual level or change in level of fibrinogen;
(y) absence or presence or actual level or change in level of fibrinolytic peptides;
(z) absence or presence or actual level or change in level of modified haemoglobin;
(aa) absence or presence or actual level or change in level of ferritin;
(bb) absence or presence or actual level or change in level of soluble intercellular adhesion molecule including soluble intercellular adhesion molecule-1;
(cc) absence or presence or actual level or change in level of heat shock proteins;
(dd) absence or presence or actual level or change in level of apoB, apoA, apoE;
(ee) absence or presence or actual level or change in level of homocysteine or parts thereof;
(ff) absence or presence or actual level or change in level of Streptococcus sp., Porphyromonas gingivalis, Helicobacter pylori or Chlamydia pneumoniae or immunological relatives thereof;
(gg) absence or presence or actual level or change in level of necrosis and platelet markers;
(hh) absence or presence or actual level or change in level of leptin;
(ii) absence or presence or actual level or change in level of vasopeptidase inhibitor of cardiac endogenous kinins;
(jj) absence or presence or actual level or change in level of heparin;
(kk) absence or presence or actual level or change in level of metalloproteinase-9;
(ll) absence or presence or actual level or change in level of metalloproteinase-1 including its tissue inhibitor;
(mm) absence or presence or actual level or change in level of angiotensin-converting enzyme;
(nn) absence or presence or change in level or an alteration in absolute amount of CD95/Apo1/Fas;
(oo) absence or presence or change in level or an alteration in absolute amount of hepatocyte growth factor;
(pp) absence or presence or change in level or an alteration in absolute amount of soluble vascular cell adhesion molecule-1;
(qq) absence or presence or change in level of or an alteration in absolute amount plasma brain natriuretic peptide;
(rr) absence or presence or change in level or an alteration in absolute amount of angiotensin II type receptor;
(ss) absence or presence or change in level or an alteration in absolute amount of endothelial constitutive nitric oxide synthase;
(tt) absence or presence or change in level of or an alteration in absolute amount glycoprotein IIIa genetic polymorphisms;
(uu) absence or presence or change in level or an alteration in absolute amount of factor VIIa;
(vv) absence or presence or change in level or an alteration in absolute amount of thrombin;
(ww) absence or presence or change in level or an alteration in absolute amount of endothelin-1;
(xx) absence or presence or change in level or an alteration in absolute amount of cardiac myofibrillar proteins;
(yy) absence or presence or change in level or an alteration in absolute amount of Fas and Fas ligand; and
(zz) absence or presence or change in level or an alteration in absolute amount of ligands thereof or binding partners thereof or nucleic acid molecules encoding same or their fragments or ligands or binding partners; and
(2) a computer readable medium that stores the code;
(3) wherein the codes determine a pattern of presence or absence of these codes is elevated as follows:
(1) the presence or actual level or elevated levels of at least five members selected from myoglobin, myosin light chain, myosin heavy chain, total creatine kinase, total creatine kinase-MB, lactate dehydrogenase, aspartate aminotransferase, cardiac troponin I, cardiac troponin T, fatty acid binding protein (FABP), glycogen phosphorylase-BB isoenzyme, α-atrial natriuretic peptide, brain natriuretic peptide, adrenomedullin, low density lipoprotein, very low density lipoprotein, high density lipoprotein and intermediate density lipoprotein, C reactive protein, serum amyloid A, P-selectin, prostaglandins, platelet-activating factor, histamine, tumor necrosis factor α, soluble TNF receptor 2, fibrin, fibrinogen, fibrinolytic peptides, modified haemoglobin, ferritin, soluble intercellular adhesion molecule including soluble intercellular adhesion molecule-1, heat shock proteins, apoB, apoA, apoE, homocysteine or parts thereof, Streptococcus sp., Porphyromonas gingivalis, Helicobacter pylori and Chlamydia pneumoniae or immunological relatives thereof, necrosis and platelet markers, leptin, vasopeptidase inhibitor of cardiac endogenous kinins, heparin, metalloproteinase-9, metalloproteinase-1 including its tissue inhibitor, angiotensin-converting enzyme, CD95/Apo1/Fas, hepatocyte growth factor, soluble vascular cell adhesion molecule-1, plasma brain natriuretic peptide, angiotensin II type receptor, endothelial constitutive nitric oxide synthase, glycoprotein IIIa genetic polymorphisms, factor VIIa, thrombin, endothelin-1, cardiac myofibrillar proteins, Fas and Fas ligand, ligands thereof or binding partners thereof or nucleic acid molecules encoding same or their fragments or ligands or binding partners;
wherein at least one of the five members is selected from myoglobin, total creatine kinase, total creatine kinase-MB, lactate dehydrogenase, cardiac troponin-I, cardiac troponin-T, brain natriuretic peptide and C reactive protein;
is indicative of an acute condition;
(2) the absence of or no change in the level of the combination of members listed in (1) is indicative of the absence of an event of the vasculature; and
(3) the presence or actual level or an elevation in at least 10 members selected from two or more of myoglobin, myosin light chain, myosin heavy chain, total creatine kinase, total creatine kinase-MB, lactate dehydrogenase, aspartate aminotransferase, cardiac troponin I, cardiac troponin T, fatty acid binding protein (FABP), glycogen phosphorylase-BB isoenzyme, α-atrial natriuretic peptide, brain natriuretic peptide, adrenomedullin, low density lipoprotein, very low density lipoprotein, high density lipoprotein and intermediate density lipoprotein, C reactive protein, serum amyloid A, P-selectin, prostaglandins, platelet-activating factor, histamine, tumor necrosis factor α, soluble TNF receptor 2, fibrin, fibrinogen, fibrinolytic peptides, modified haemoglobin, ferritin, soluble intercellular adhesion molecule including soluble intercellular adhesion molecule-1, heat shock proteins, apoB, apoA, apoE, homocysteine or parts thereof, Streptococcus sp., Porphyromonas gingivalis, Helicobacter pylori and Chlamydia pneumoniae or immunological relatives thereof, necrosis and platelet markers, leptin, vasopeptidase inhibitor of cardiac endogenous kinins, heparin, metalloproteinase-9, metalloproteinase-1 including its tissue inhibitor, angiotensin-converting enzyme, CD95/Apo1/Fas, hepatocyte growth factor, soluble vascular cell adhesion molecule-1, plasma brain natriuretic peptide, angiotensin II type receptor, endothelial constitutive nitric oxide synthase, glycoprotein IIIa genetic polymorphisms, factor VIIa, thrombin, endothelin-1, cardiac myofibrillar proteins, Fas and Fas ligand, ligands thereof or binding partners thereof or nucleic acid molecules encoding same or their fragments or ligands or binding partners is indicative of a chronic condition.

19. A method of treating a subject with a vasculature related tissue injury, vasculature insufficiency or infarct or related condition wherein the size of the vasculature related tissue injury, vasculature insufficiency or infarct is determined by the formula:—
$$Is = \frac{{\int }_{0}^{t} f ( t ) ⅆ t \times Bw \times Kw}{Ed \times Kr}$$
wherein
Is is a vasculature related injury, vasculature insufficiency or infarct size
f(t)dt is the rate of release of a member in a biological sample, said member being present, absent, elevated or otherwise activated in a subject following a cardiovascular condition or event leading to the vasculature related injury, vasculature insufficiency or infarct;
Bw is the body weight of the subject;
Kw is the proportion of the body weight into which the member is released;
Ed is the rate of removal of the member from evaluation; and
Kr is the total amount of member released divided by the amount of the member released from the vasculature related injury, vasculature insufficiency or infarcted tissue;
said method comprising contacting a biological sample from a subject to be tested wherein said biological sample comprises one or more members which are present, absent, elevated or otherwise activated in a subject following said condition or event or a condition or event otherwise associated with a aberration wherein said members are selected from two or more of myoglobin, myosin light chain, myosin heavy chain, total creatine kinase, total creatine kinase-MB, lactate dehydrogenase, aspartate aminotransferase, cardiac troponin I, cardiac troponin T, fatty acid binding protein (FABP), glycogen phosphorylase-BB isoenzyme, α-atrial natriuretic peptide, brain natriuretic peptide, adrenomedullin, low density lipoprotein, very low density lipoprotein, high density lipoprotein and intermediate density lipoprotein, C reactive protein, serum amyloid A, P-selectin, prostaglandins, platelet-activating factor, histamine, tumor necrosis factor α, soluble TNF receptor 2, fibrin, fibrinogen, fibrinolytic peptides, modified haemoglobin, ferritin, soluble intercellular adhesion molecule including soluble intercellular adhesion molecule-1, heat shock proteins, apoB, apoA, apoE, homocysteine or parts thereof, Streptococcus sp., Porphyromonas gingivalis, Helicobacter pylori and Chlamydia pneumoniae or immunological relatives thereof, necrosis and platelet markers, leptin, vasopeptidase inhibitor of cardiac endogenous kinins, heparin, metalloproteinase-9, metalloproteinase-1 including its tissue inhibitor, angiotensin-converting enzyme, CD95/Apo1/Fas, hepatocyte growth factor, soluble vascular cell adhesion molecule-1, plasma brain natriuretic peptide, angiotensin II type receptor, endothelial constitutive nitric oxide synthase, glycoprotein IIIa genetic polymorphisms, factor VIIa, thrombin, endothelin-1, cardiac myofibrillar proteins, Fas and Fas ligand, ligands thereof or binding partners thereof or nucleic acid molecules encoding same or their fragments or ligands or binding partners and contacting said biological sample with one or more antibodies or immunological equivalents thereof capable of binding to said one or more members in the biological sample and wherein the pattern of interaction between said members and antibodies including the absence of interaction is indicative of the size of the vasculature related injury, vasculature insufficiency or an infarct and then effecting a suitable treatment regimen.

20. A method of determining the presence or absence of a condition or event of the vasculature or determining a risk of a condition or event occurring, said method comprising obtaining a biological sample from a subject to be tested wherein said biological sample comprises one or more members which are present, absent, elevated or otherwise activated or up or down regulated in a subject prior to, during or following said condition or event and contacting said biological sample with a second set of members wherein one or more of said second set of members are binding partners to one or more of said first set of members and wherein the pattern of interaction between said first and second sets of members including the absence of interaction is indicative of said condition or event or the risk of development of same.

21. The method of claim 20 wherein members in the biological sample are selected from two or more of myoglobin, myosin light chain, myosin heavy chain, total creatine kinase, total creatine kinase-MB, lactate dehydrogenase, aspartate aminotransferase, cardiac troponin I, cardiac troponin T, fatty acid binding protein (FABP), human heart-type, glycogen phosphorylase-BB isoenzyme, α-atrial natriuretic peptide, brain natriuretic peptide, adrenomedullin, low density lipoprotein, very low density lipoprotein, high density lipoprotein and intermediate density lipoprotein, C reactive protein, serum amyloid A, P-selectin, prostaglandins, platelet-activating factor, histamine, tumor necrosis factor α, soluble TNF receptor 2, fibrin, fibrinogen, fibrinolytic peptides, modified haemoglobin, ferritin, soluble intercellular adhesion molecule including soluble intercellular adhesion molecule-1, heat shock proteins, apoB, apoA, apoE, homocysteine or parts thereof, Streptococcus sp., Porphyromonas gingivalis, Helicobacter pylori and Chlamydia pneumoniae or immunological relatives thereof, necrosis and platelet markers, leptin, vasopeptidase inhibitor of cardiac endogenous kinins, heparin, metalloproteinase-9, metalloproteinase-1 including its tissue inhibitor, angiotensin-converting enzyme, CD95/Apo1/Fas, hepatocyte growth factor, soluble vascular cell adhesion molecule-1, plasma brain natriuretic peptide, angiotensin II type receptor, endothelial constitutive nitric oxide synthase, glycoprotein IIIa genetic polymorphisms, factor VIIa, thrombin, endothelin-1, cardiac myofibrillar proteins, Fas and Fas ligand, ligands thereof or binding partners thereof or nucleic acid molecules encoding same or their fragments or ligands or binding partners.

22. The method of claim 20 wherein the binding partners of the members of the biological sample are immunointeractive molecules.

23. The method of claim 22 wherein the immunointeractive molecules are antibodies.

24. The method claim 20 wherein the second set of members are immobilized to a solid support.

25. The method of claim 24 wherein binding of members to binding partners is detected by a labeled antibody to the member in the biological sample.

26. The method of claim 25 wherein the binding of a binding member to a binding partner is detected by a method selected from:—
(i) biotinylation of all plasma proteins, which are then bound to an antibody microarray and then to a streptavidin-AP/HRP conjugate;
(ii) fluorescent labeling of all plasma proteins;
(iii) fluorescently-labeled antibodies specific for a different epitope; and
(iv) concentrations of plasma markers determined using dilutions of immobilized antibodies.
